(19) United States
(12) Patent Application Publication
(10) Pub. No.: US 2024/0247207 A1
(43) Pub. Date: Jul. 25, 2024

(54) MALTOSIDE-, LACTOBIONAMIDE-, MONOGLUCOSIDE-, BRANCHED DIGLUCOSIDE-, SULFOBETAINE-, SULFATE- OR AMINOOXIDE-BASED PERFLUORINATED DETERGENTS AND THEIR USE IN MEMBRANE-PROTEINS APPLICATIONS

(71) Applicants: AVIGNON UNIVERSITE, AVIGNON (FR); CENTRE NATIONAL DE LA RECHERCHE SCIENTIFIQUE, PARIS (FR); UNIVERSITE DE MONTPELLIER, MONTPELLIER (FR); ECOLE NATIONALE SUPERIEURE DE CHIMIE DE MONTPELLIER, MONTPELLIER (FR)

(72) Inventors: GRÉGORY DURAND, VILLENEUVE LES AVIGNON (FR); MOHEDDINE WEHBIE, PARIS (FR)

(21) Appl. No.: 18/288,397

(22) PCT Filed: Apr. 29, 2022
(86) PCT No.: PCT/EP2022/061455

§ 371 Date: Oct. 26, 2023

(30) Foreign Application Priority Data
Apr. 29, 2021 (EP) 21305551.0

(51) Int. Cl. C11D 1/00; C07C 317/28; C07H 15/06; C07H 15/18; C07K 14/245

(52) U.S. Cl. CPC C11D 1/004; C07C 317/28; C07H 15/06; C07H 15/18; C07K 14/245; C07C 2601/14

(57) ABSTRACT

The present invention relates to new amphiphilic perfluorinated compounds and their use as a detergent for extracting membrane-proteins or synthesizing membrane-proteins in acellular system. It also relates to a method for extracting a membrane-protein from a biological sample using such compounds.

### TECHNICAL FIELD

The present invention relates to new amphiphilic perfluorinated compounds and their use as a detergent for extracting membrane-proteins or synthesizing membrane-proteins in acellular system. It also relates to a method for extracting a membrane-protein from a biological sample using such compounds.

### TECHNICAL BACKGROUND

The proteins located at the cell membranes, called “membrane-proteins”, hold a crucial role in the relay of signals between the cell's internal and external environments or in communication between cells. These proteins account for about one third of the proteins encoded within the human genome and more than 50% of the therapeutic targets, which makes their study a prominent research field. Current breakthroughs in this research field are sustained by the development of tools and technics for the extraction of a membrane-protein. Extraction of a membrane-protein comprises its solubilization, which enables the membrane-protein to be isolated from the cell membrane, and its stabilization which aims at maintaining its native structure and function.

Due to their delipidating nature, classical detergents (or surfactants) are commonly used for extracting membrane-proteins. For instance, DDM (n-dodecyl-β-D-maltoside) is one of the most common detergents used for membrane-protein extraction. U.S. Pat. Nos. 5,674,987 and 5,763,586 relate to detergents prepared from the reaction of a cycloalkyl aliphatic alcohol with a saccharide, and their use for extracting proteins from naturally occurring membranes. Various detergents are disclosed, each of them being composed of a saccharide moiety, in particular a maltoside or a glucoside, and a non-substituted cycloalkyl moiety, both moieties being separated by a linear alkylene chain. However, such detergents cannot generally stabilize membrane-proteins due to their strong delipidating nature.

Some other detergents, which have proven to have a softer delipidating nature, are generally able to stabilize but not to efficiently solubilize membrane-proteins. It is therefore usually necessary to successively use two detergents for extracting a membrane-protein: a first detergent which solubilizes the protein and then a second detergent which stabilizes the solubilized protein.

Frotscher et al. (Angew. Chem. Int. Ed. 2015, 54, 5069-5073) disclose fluorinated detergents for membrane-protein applications. The study focuses on detergent (1H, 1H, 2H, 2H-Perfluorooctyl)-β-D-Maltopyranoside (F₆OM), composed of a maltose unit linked to a linear perfluorinated hexyl chain through an ethyl linker. The study demonstrates that such a detergent can induce solubilization of lipid vesicles, which are composed of long chain phospholipids, and can also chaperone the functional refolding of outer membrane phospholipase A (OmpLA) into these vesicles. However, low extraction yields are obtained with F₆OM. In addition, the ability to solubilize and stabilize cannot be extended to just any membrane-proteins.

Thus, there remains a need to provide new detergents which are able to efficiently solubilize and stabilize membrane-proteins, and more specifically membrane-proteins which have not been extracted satisfactorily by already known detergents.

### SUMMARY OF THE INVENTION

In this respect, the inventors have developed new perfluorinated detergents, and have demonstrated that these detergents were able to efficiently solubilize, stabilize, and consequently extract, membrane-proteins, such as Bacteriorhodopsin or FhuA (Ferrichrome outer membrane transporter/phase receptor). Comparative tests have shown that such compounds were a good alternative to F₆OM, but also DDM. Besides extraction, other membrane-protein applications can be contemplated, in particular the acellular synthesis of membrane-proteins.

Thus, the present invention thus relates to a compound represented by the following formula (I):

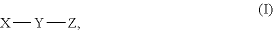

wherein:
X is a polar moiety chosen from a maltoside, a lactobionamide, a glucoside, a sulfobetaine, an aminoxide, a sulfate, and a branched diglucoside moiety;
Y is a (C₁-C₁₂) aliphatic linker optionally comprising one or more heteroatomic groups chosen each independently from —O—, —NH—, —S—, —C(O)—, —NH—C(O)—, —C(O)—NH—, —C(O)—O—, —O—C(O)—, —NH—C(O)—O—, —O—C(O)—NH— and a triazole; and
Z is chosen from a perfluorinated (C₃-C₁₂)cycloalkyl and a branched perfluorinated (C₃-C₁₂)alkyl.

In a particular embodiment, said compound of formula (I) is such that:
X is a maltoside or lactobionamide moiety, preferably a maltoside moiety;
Y is a (C₁-C₁₂) aliphatic linker; and
Z is chosen from a perfluorinated (C₃-C₁₂)cycloalkyl and a branched perfluorinated (C₃-C₁₂)alkyl.

In another particular embodiment, X is a maltoside, a lactobionamide or a sulfobetaine moiety, preferably a maltoside or a lactobionamide moiety, more preferably a maltoside moiety.

In another particular embodiment, Y is a (C₃-C₈)alkylene linker. Preferably, Y is selected from a propylene, a butylene and a pentylene.

In another particular embodiment, Z is a perfluorinated (C₃-C₁₂)cycloalkyl, preferably a perfluorinated (C₅-C₇)cycloalkyl. More preferably, Z is a perfluorinated cyclohexyl.

In another particular embodiment, Z is a perfluorinated isoheptyl or a perfluorinated isopentyl.

In a particular embodiment, said compound of formula (I) is such that:
X is a maltoside, a lactobionamide or a sulfobetaine moiety, preferably a maltoside or a lactobionamide moiety, more preferably a maltoside moiety;
Y is a (C₃-C₅)alkylene linker; and
Z is a perfluorinated cyclohexyl.

In a preferred embodiment, a compound of the invention is of formula (II):

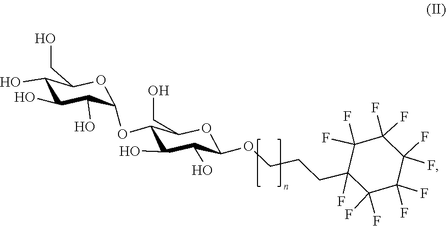

in which n is 1, 2 or 3.

In another preferred embodiment, a compound of the invention is of formula (III):

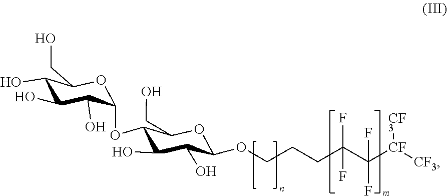

in which n is 1, 2 or 3, and m is 1 or 2.

Preferably, a compound of the invention is selected from the group consisting of:
3-(perfluorocyclohexyl)-propoxy-4-O—(α-D-glucopyranosyl)-β-D-glucopyranoside,
4-(perfluorocyclohexyl)-butoxy-4-O—(α-D-glucopyranosyl)-β-D-glucopyranoside,
5-(perfluorocyclohexyl)-pentoxy-4-O—(α-D-glucopyranosyl)-β-D-glucopyranoside,
3-(perfluoroisopentyl)-propoxy-4-O—(α-D-glucopyranosyl)-β-D-glucopyranoside,
3-(perfluoroisoheptyl)-propoxy-4-O—(α-D-glucopyranosyl)-β-D-glucopyranoside,
5-(perfluoroisopentyl)-pentoxy-4-O—(α-D-glucopyranosyl)-β-D-glucopyranoside,
3-(perfluorocyclohexyl)-N-propane Octahydroxy lactobionamide, and
3-(dimethyl(5-(perfluorocyclohexyl)pentyl)ammonio)propane-1-sulfonate.

The present invention also relates to a detergent composition comprising at least one compound of formula (I) as defined herein.

The present invention further relates to the use of a compound as defined herein, or a detergent composition comprising the same, as a detergent for extracting membrane-proteins or synthesizing membrane-proteins in acellular system. In a particular embodiment, said membrane-protein is Bacteriorhodopsin or FhuA.

Another object of the invention is an in-vitro method for extracting a membrane-protein from a biological sample comprising the following steps:
(a) contacting a compound as defined herein or a detergent composition as defined herein, with a biological sample comprising a membrane-protein; and
(b) recovering a membrane-protein.

### BRIEF DESCRIPTION OF THE FIGURES

FIG. 1: Kinetics of 100 μM POPC LUVs solubilization (A) by 10.06 mM of compound 5a, 7.16 mM of compound 5b and 5.69 mM of compound 5c and (B) by 8.62 mM of compound 8a and 5.46 mM of compound 8c at 25° C. as monitored in terms of the light scattering intensity recorded at an angle of 90°.

FIG. 2: SDS-PAGE of E. coli membrane extracts upon exposure to compound 5a, DDM, F₆OM and F₆OPC at various concentrations (CMC+1 mM, CMC+2 mM, CMC+5 mM and CMC+10 mM).

FIG. 3: Graphical representation of protein-extraction yields when using compound 5a, 5c, DDM, F₆OM, F₆OPC relative to the yield obtained when no surfactant was added (only buffer).

Data are mean values from three experiments.

### DETAILED DESCRIPTION OF THE INVENTION

The compound of the present invention consists of three moieties, namely X, Y, and Z, wherein X and Z are linked to each other through the linker Y. Typically, the bond between X and Y and the bond between Y and Z are covalent bonds.

According to the present invention, X denotes a polar moiety (or equivalently, a “polar head”). Said polar moiety may be zwitterionic, ionic or non-ionic. More specifically, X is chosen from the following polar moieties: a maltoside, a lactobionamide, a glucoside, a sulfobetaine, an aminoxide, a sulfate, and a branched diglucoside moiety.

The maltoside moiety may be an alpha- or beta-maltoside moiety.

In a particular embodiment, the maltoside moiety is a beta-maltoside moiety. Such maltoside moiety can be represented by the following formula (X-I):

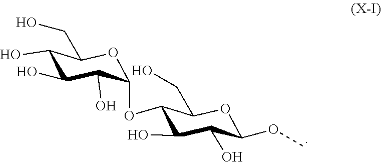

In another particular embodiment, the maltoside moiety is an alpha-maltoside moiety. Such maltoside moiety can be represented by the following formula (X-I′):

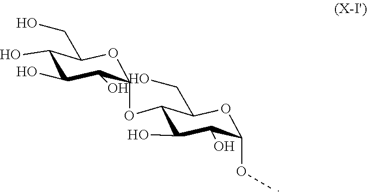

Preferably, the maltoside moiety is represented by the formula (X-I).

A lactobionamide moiety can be represented by the following formula (X-II):

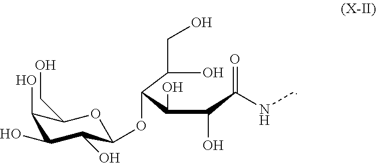

The glucoside moiety may be an alpha- or beta-glucoside moiety.

In a particular embodiment, the glucoside moiety is a beta-glucoside moiety. Such glucoside moiety can be represented by the following formula (X-III):

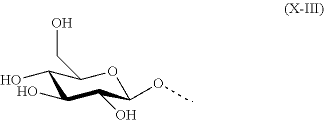

In another particular embodiment, the glucoside moiety is an alpha-glucoside moiety. Such glucoside moiety can be represented by the following formula (X-III′):

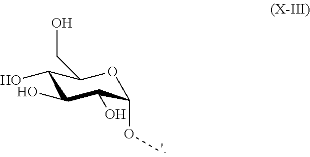

Preferably, the glucoside moiety is represented by the formula (X-III).

A sulfobetaine moiety can be represented by the following formula (X-IV):

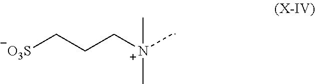

An aminoxide moiety can be represented by the following formula (X-V):

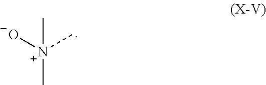

A sulfate moiety can be represented by the following formula (X-VI):

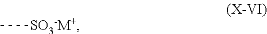

wherein M⁺ is an organic or inorganic cation.

A particular organic cation is NR4 wherein each R is independently a methyl, an ethyl, a propyl or a butyl.

Preferably, M⁺ is an inorganic cation, such as an alkaline metal cation (e.g. sodium, lithium, or potassium) or a transition metal cation (e.g. silver). More preferably, M⁺ is a sodium cation.

A branched diglucoside moiety can be represented by the following formula (X-VII):

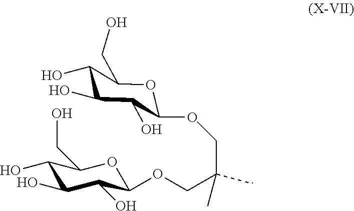

As used herein, the symbol “- - - -” represents the bond by which a moiety is attached to the remainder of the molecule. In formulae (X-I) to (X-VII), said symbol represents the bond between the polar moiety X as represented and Y in formula (I). For instance, in formula (X-I), said symbol represents the bond between the maltoside moiety as represented and Y in formula (I). For instance, in formula (X-II), said symbol represents the bond between the lactobionamide moiety as represented and Y in formula (I). For instance, in formula (X-III), said symbol represents the bond between the glucoside moiety as represented and Y in formula (I). For instance, in formula (X-IV), said symbol represents the bond between the sulfobetaine moiety as represented and Y in formula (I). For instance, in formula (X-V), said symbol represents the bond between the aminoxide moiety as represented and Y in formula (I). For instance, in formula (X-VI), said symbol represents the bond between the sulfate moiety as represented and Y in formula (I). For instance, in formula (X-VII), said symbol represents the bond between the branched diglucoside moiety as represented and Y in formula (I).

In a particular embodiment, X is a maltoside, a lactobionamide, or a sulfobetaine moiety. In a preferred embodiment, X is a maltoside or a lactobionamide moiety. In a more preferred embodiment, X is a maltoside moiety.

According to the present invention, Y is a (C₁-C₁₂) aliphatic linker, optionally comprising one or more heteroatomic groups chosen each independently from —O—, —NH—, —S—, —C(O)—, —NH—C(O)—, —C(O)—NH—, —C(O)—O—, —O—C(O)—, —NH—C(O)—O—, —O—C(O)—NH—, and a triazole.

In a particular embodiment, Y is a (C₁-C₁₂) aliphatic linker, such as a (C₂-C₁₂), (C₃-C₁₀), (C₄-C₉), or (C₅-C₈) aliphatic linker, in particular a (C₁-C₁₀), (C₃-C₁₀), (C₃-C₈), (C₃-C₆), or (C₃-C₈) aliphatic linker, preferably a (C₃-C₈) aliphatic linker.

The term “(C₁-C₁₂) aliphatic linker” refers to a linear or branched (preferably linear), saturated or unsaturated, acyclic, non-aromatic hydrocarbon divalent chain having 1 to 12 carbon atoms.

The (C₁-C₁₂) aliphatic linker may in particular be a (C₁-C₁₂) alkylene linker. As used herein, a “(C₁-C₁₂) alkylene linker” is a linear or branched (preferably linear), saturated, acyclic, non-aromatic hydrocarbon divalent chain having 1 to 12 carbon atoms.

The alkylene linker may in particular be represented by the formula —(CH₂)_{q}— wherein q is an integer from 1 to 12.

Examples of alkylene linker are a methylene (e.g. —CH₂—), an ethylene (e.g. —(CH₂)₂—), a propylene (e.g. —(CH₂)₃—), a butylene (e.g. —(CH₂)₄—), a pentylene (e.g. —(CH₂)₅—), a hexylene (e.g. —(CH₂)₆—), a heptylene (e.g. —(CH₂)₇—), an octylene (e.g. —(CH₂)₈—), a nonylene (e.g. —(CH₂)₉—), a decylene (e.g. —(CH₂)₁₀—), an undecylene (e.g. —(CH₂)₁₁—), or a dodecylene (e.g. —(CH₂)₁₂—).

Y may in particular be a (C₁-C₁₀), (C₃-C₁₀), (C₃-C₈), (C₃-C₆), or (C₃-C₈) alkylene linker.

In a more particular embodiment, Y is a (C₃-C₈) alkylene linker (i.e. an alkylene linker having 3 to 5 carbon atoms).

Preferably, Y is selected from a propylene (e.g. —(CH₂)₃—), a butylene (e.g. —(CH₂)₄—) and a pentylene (e.g. —(CH₂)₅—).

Y as defined above may comprise or may not comprise one or more heteroatomic groups chosen each independently from —O—, —NH—, —S—, —C(O)—, —NH—C(O)—, —C(O)—NH—, —C(O)—O—, —O—C(O)—, —NH—C(O)—O—, —O—C(O)—NH—, and a triazole.

In a particular embodiment, Y is a (C₁-C₁₂) aliphatic linker as defined above that does not comprise any of said heteroatomic groups.

In another particular embodiment, Y is such as defined above and further comprises one or more (for instance one, two, or three, preferably one or two, more preferably one) heteroatomic groups chosen each independently from —O—, —NH—, —S—, —C(O)—, —NH—C(O)—, —C(O)—NH—, —C(O)—O—, —O—C(O)—, —NH—C(O)—O—, —O—C(O)—NH—, and a triazole.

It is understood that said heteroatomic groups can be at any position of the aliphatic linker Y.

For instance, a heteroatomic group may be at one end of the aliphatic linker Y. In such case, the heteroatomic group links the polar moiety X and the aliphatic linker Y or the heteroatomic group links the aliphatic linker Y and the perfluorinated group Z.

For instance, a heteroatomic group may be at each end of the aliphatic linker Y. In such case, one heteroatomic group links the polar moiety X and the aliphatic linker Y and another one heteroatomic group links the aliphatic linker Y and the perfluorinated group Z.

Alternatively, or in addition, one or more heteroatomic groups may be at any position within the aliphatic linker Y (i.e. any position interrupting the aliphatic linker Y).

The triazole group can be represented by any one of the following formulae:

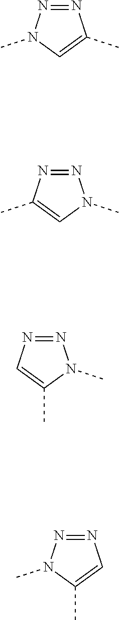

Preferably, the triazole group is represented by any one of the following formulae:

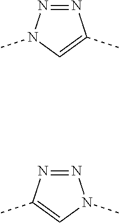

As used herein, the symbol “- - - -” represents the bond by which the group is attached to the remainder of the molecule.

It is also understood that, when said one or more heteroatomic groups contain carbon atoms (e.g. —C(O)—, —NH—C(O)—, —C(O)—NH—, —C(O)—O—, —O—C(O)—, —NH—C(O)—O—, —O—C(O)—NH—, or the two ring carbon atoms of the triazole group), such carbon atoms are included within the total number of carbon atoms of the aliphatic linker Y. For example, the following linker Y is a C₆ aliphatic linker comprising two heteroatomic groups, —NH—C(O)— and —S—:

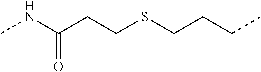

Examples of C₁-C₁₂ aliphatic linkers Y comprising one or more heteroatomic groups include, but are not limited to:

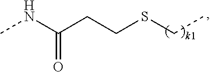

wherein k1 is an integer from 0 to 9, or

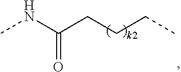

wherein k2 is an integer from 1 to 9.

In a particular embodiment, Y is a C₁-C₁₂ alkylene linker (preferably C₁-C₆ alkylene linker), optionally comprising one or two heteroatomic groups chosen each independently from —S—, —NH—C(O)—, and —C(O)—NH—.

According to the present invention, Z is chosen from a perfluorinated (C₃-C₁₂)cycloalkyl and a branched perfluorinated (C₃-C₁₂)alkyl.

As used herein, a “(C₃-C₁₂)cycloalkyl group” refers to a saturated, mono-, bi-, or tri-cyclic, optionally bridged, hydrocarbon chain having 3 to 12 carbon atoms. Examples of C₃-C₁₂ cycloalkyl group include, but are not limited to, cyclopropyl, cyclobutyl, cyclopentyl, cyclohexyl, cycloheptyl, cyclooctyl, cyclodecyl or cyclododecyl.

As used herein, a “branched (C₃-C₁₂)alkyl” refers to a branched, saturated, acyclic hydrocarbon chain having 3 to 12 carbon atoms. Examples of C₃-C₁₂ branched alkyl group include, but are not limited to, isopropyl, isobutyl, isopentyl (also called “isoamyl”), isohexyl, isoheptyl, isooctyl, isononyl, isodecyl, isoundecyl, isododecyl, sec-butyl, tert-butyl, neopentyl, or tert-pentyl.

As used herein, a “perfluorinated” group refers to a group wherein all the hydrogen atoms of said group have been replaced by fluorine atoms.

In a particular embodiment, Z is a perfluorinated (C₅-C₇)cycloalkyl (i.e. a cycloalkyl having from 5 to 7 carbon atoms). In a more particular embodiment, Z is a perfluorinated cyclohexyl or cyclopentyl. Preferably, Z is a perfluorinated cyclohexyl.

In another particular embodiment, Z is a branched perfluorinated (C₄-C₉)alkyl (i.e. a branched perfluorinated alkyl having 4 to 9 carbon atoms). Preferably, Z is a perfluorinated isoheptyl or a perfluorinated isopentyl, more preferably a perfluorinated isopentyl.

In a particular embodiment, the compound of formula (I) is such that:
X is a maltoside or a lactobionamide moiety;
Y is a (C₁-C₁₂)aliphatic linker; and
Z is chosen from a perfluorinated (C₃-C₁₂)cycloalkyl and a branched perfluorinated (C₃-C₁₂)alkyl.

In another particular embodiment, said compound of formula (I) is such that:
X is a maltoside, a lactobionamide or a sulfobetaine moiety, preferably a maltoside or a lactobionamide moiety, more preferably a maltoside moiety;
Y is a (C₃-C₈)alkylene linker; and
Z is a perfluorinated cyclohexyl.

In a particular embodiment, X is a lactobionamide, Y is a (C₃-C₈) alkylene linker, and Z is a branched perfluorinated (C₄-C₉)alkyl or a perfluorinated (C₅-C₇)cycloalkyl. In such embodiment, Z is preferably a perfluorinated cyclohexyl, a perfluorinated isoheptyl or a perfluorinated isopentyl.

In a preferred embodiment, the compound of the invention is represented by the following formula (II):

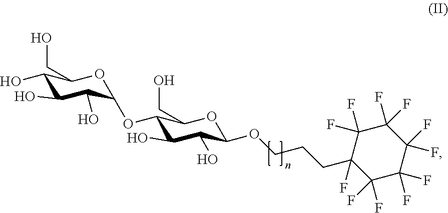

in which n is 1, 2 or 3.

In another preferred embodiment, the compound of the invention is represented by the following formula (III):

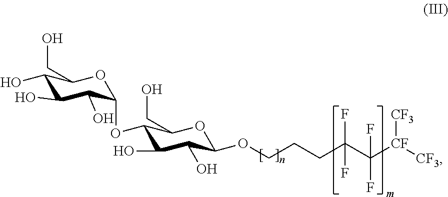

in which n is 1, 2 or 3, and m is 1 or 2 (preferably m is 1).

In a more preferred embodiment, the compound of the invention is a compound of formula (III) wherein:
n is 1 and mis 1;
n is 1 and m is 2; or
n is 3 and m is 1.

In a further preferred embodiment, the compound of the invention is a compound of formula (III) wherein n is 1 or 3, and m is 1.

In a particular embodiment, the compound of the invention is represented by the following formula (IV):

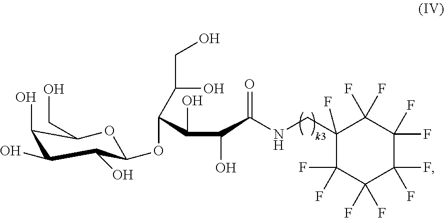

wherein k3 is an integer from 1 to 12, preferably from 1 to 5, more preferably k3 is 3.

In another particular embodiment, the compound of the invention is represented by the following formula (V):

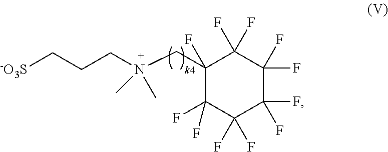

wherein k4 is an integer from 1 to 12, preferably from 1 to 5, more preferably k4 is 5.

In another particular embodiment, the compound of the invention is represented by the following formula (VI):

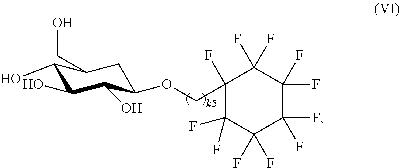

wherein k5 is an integer from 1 to 12, preferably from 1 to 5.

In another particular embodiment, the compound of the invention is represented by the following formula (VII):

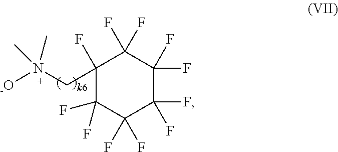

wherein k6 is an integer from 1 to 12, preferably from 1 to 5.

In another particular embodiment, the compound of the invention is represented by the following formula (VIII):

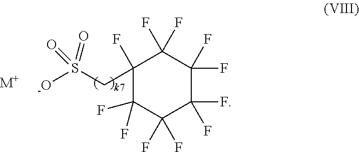

wherein:
k7 is an integer from 1 to 12, preferably from 1 to 5, and
M⁺ is an organic or inorganic cation, preferably an inorganic cation, more preferably an alkaline metal cation such as sodium.

In another particular embodiment, the compound of the invention is represented by the following formula (IX):

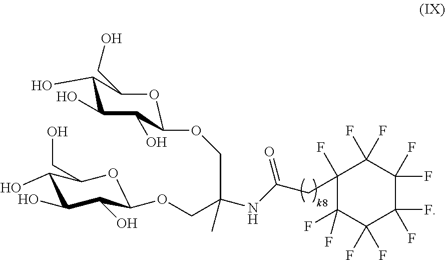

wherein k8 is an integer from 1 to 12, preferably from 1 to 8, more preferably from 3 to 6.

In another particular embodiment, the compound of the invention is represented by the following formula (X):

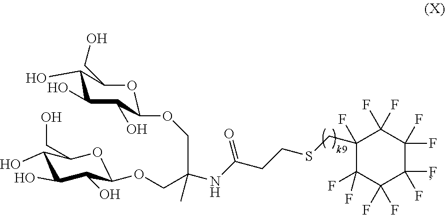

wherein k9 is an integer from 0 to 9, preferably from 1 to 6.

In a particular embodiment, the compound of the invention is represented by formula (XI) or (XII):

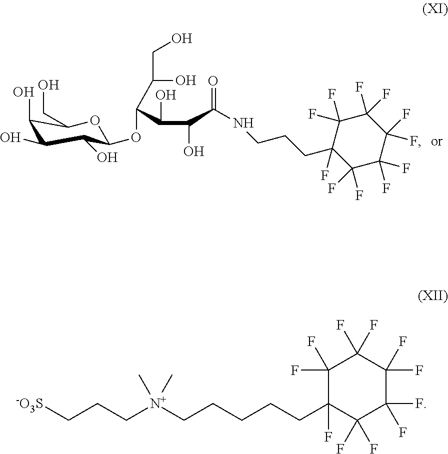

Advantageously, compounds of the invention, in particular compounds of formulae (II), (III), (IV), (V), (VII), (VIII), (IX) or (X) wherein the linker Y has at most 5 or 6 carbon atoms, have a high solubility.

In a particular embodiment, the compound of the invention is selected from the group consisting of:
3-(perfluorocyclohexyl)-propoxy-4-O—(α-D-glucopyranosyl)-β-D-glucopyranoside,
4-(perfluorocyclohexyl)-butoxy-4-O—(α-D-glucopyranosyl)-β-D-glucopyranoside,
5-(perfluorocyclohexyl)-pentoxy-4-O—(α-D-glucopyranosyl)-β-D-glucopyranoside,
3-(perfluoroisopentyl)-propoxy-4-O—(α-D-glucopyranosyl)-β-D-glucopyranoside,
3-(perfluoroisoheptyl)-propoxy-4-O—(α-D-glucopyranosyl)-β-D-glucopyranoside,
5-(perfluoroisopentyl)-pentoxy-4-O—(α-D-glucopyranosyl)-β-D-glucopyranoside,
3-(perfluorocyclohexyl)-N-propane Octahydroxy lactobionamide, and
3-(dimethyl(5-(perfluorocyclohexyl)pentyl)ammonio)propane-1-sulfonate.

Preferably, the compound of the invention is selected from the group consisting of:
3-(perfluorocyclohexyl)-propoxy-4-O—(α-D-glucopyranosyl)-β-D-glucopyranoside,
4-(perfluorocyclohexyl)-butoxy-4-O—(α-D-glucopyranosyl)-β-D-glucopyranoside,
5-(perfluorocyclohexyl)-pentoxy-4-O—(α-D-glucopyranosyl)-β-D-glucopyranoside,
3-(perfluorocyclohexyl)-N-propane Octahydroxy lactobionamide, and
3-(dimethyl(5-(perfluorocyclohexyl)pentyl)ammonio)propane-1-sulfonate.

More preferably, the compound of the invention is selected from the group consisting of:
3-(perfluorocyclohexyl)-propoxy-4-O—(α-D-glucopyranosyl)-β-D-glucopyranoside,
4-(perfluorocyclohexyl)-butoxy-4-O—(α-D-glucopyranosyl)-β-D-glucopyranoside, and
5-(perfluorocyclohexyl)-pentoxy-4-O—(α-D-glucopyranosyl)-β-D-glucopyranoside.

The compounds of the invention can be prepared based on any suitable method known to the skilled artisan. In particular, the compounds of the invention can be prepared as detailed in the examples. More specifically, the compounds of the invention may be prepared by reacting a 0-protected maltose with an alkenol in the presence of a Lewis acid (such as BF3), and then reacting the obtained compound with a perfluorocycloalkyl or branched perfluoroalkyl halide in the presence of a radical initiator (such as BEt3 or AIBN). The obtained compound can be reduced for instance by using hydrogen and a palladium catalyst. O-deprotection of the maltoside moiety of the reduced compound can then be carried out by any suitable deprotection reagent known to the skilled artisan.

A similar process can be used for lactobionamide derivatives, starting from a O-protected lactobionate and an alkenamine.

Another object of the invention is a detergent composition comprising at least one compound of formula (I) as defined herein.

Such composition may further comprise other detergents known to the skilled artisan. Examples of such known detergents include, but are not limited to, n-dodecyl-β-D-maltoside, (1H, 1H, 2H, 2H-Perfluorooctyl)-β-D-Maltopyranoside, (1H, 1H, 2H, 2H-Perfluorooctyl)phosphocholine, 2,6-Dimethyl-4-Heptyl-β-D-Maltopyranoside, Decyldimethylamine-N-Oxide, Lauryl dimethylamine-N-Oxide, Cyclohexyl-Methyl-β-D-Maltoside, 2-Cyclohexyl-1-Ethyl-β-D-Maltoside, 3-Cyclohexyl-1-Propyl-β-D-Glucoside, 4-Cyclohexyl-1-Butyl-β-D-Glucoside, 4-Cyclohexyl-1-Butyl-β-D-Maltoside, 5-Cyclohexyl-1-Pentyl-β-D-Maltoside, 6-Cyclohexyl-1-Hexyl-β-D-Maltoside, 7-Cyclohexyl-1-Heptyl-β-D-Maltoside, or 3-Dodecyloxypropyl-1-β-D-Maltopyranoside.

The compounds of the invention or the composition of the invention are particularly well-suited for a use as a detergent (or “surfactant”) for solubilizing, stabilizing, reconstituting, and/or purifying, and more particularly extracting membrane-proteins.

The term “membrane-protein”, as used herein, encompasses any type of membrane protein found in any living being. The membrane-protein may in particular be an integral membrane-protein (such as transmembrane protein, mono-, bi- or poly-topic membrane-protein), a peripheral membrane-protein, or a lipid-anchored protein.

Examples of membrane-proteins include, but are not limited to, outer membrane phospholipase A (OmpLA), FhuA, degenerin sodium channel, P2X receptor, acid sensing ion channels, voltage-gated ion channels, nicotinic acetylcholine receptor, M2 channel of influenza A virus, MscL, MscS, MscM, MscK, aquaporine, ClyA, α-HL, P-type ATPase, Light-driven pumps such as Bacteriorhodopsin, ATP-binding cassette transporters such as glycoprotein P or flippase MsbA, Protein Translocation Channel such as SecYEβ or SecA-SecYEG, complex I NaDH:ubiquinone reductase, complex II succinate:ubiquinone reductase, complex III ubiquinol:cytochrome c reductase, complex IV cytochrome c oxidase, Light Harvesting Complexes, PSI, PSII, rhodopsin, adenosine receptor A2a, adrenergic receptors, Rhomboid protease GlpG, thiol oxidases, Membrane Associated Proteins in eicosanoid and glutathione metabolism) (MAPEG proteins), or carnitine palmitoyl transferase.

In a particular embodiment, the membrane-protein is Bacteriorhodopsin or FhuA.

The compounds of the present invention can also be used for synthesizing membrane-proteins in acellular system (or “acellular medium”). The compounds of the invention can in particular be used to solubilize, stabilize, and favor the refolding of the membrane-proteins produced in acellular system. The compounds of the invention can also be used to insert the membrane-proteins thus produced into a preformed lipid bilayer.

The synthesis of membrane-proteins in acellular system (also called “acellular synthesis” or “in vitro synthesis”) refers to the synthesis of membrane-proteins in a reaction mixture comprising defined biological extracts and/or reagents. The reaction mixture comprises at least one compound of formula (I) as a detergent, and can further comprise a template for the production of the membrane-protein (typically DNA or mRNA), amino acids as monomers, and cofactors, enzymes, salts, and other reagents that are necessary for the synthesis. Such synthetic reaction systems are well known in the art and have been described in the literature, for instance in Sachse et al. FEBS Letters, 2014, 588, 17, 2774-2781. The acellular synthesis reaction can be carried out discontinuously, as a continuous flow or a semi-continuous flow, as is known in the art.

Another object of the present invention is an in-vitro method for extracting a membrane-protein from a biological sample comprising the following steps:
(a) contacting a compound as defined herein or a detergent composition as defined herein, with a biological sample comprising a membrane-protein; and
(b) recovering a membrane-protein.

As used herein, the expression “extracting membrane-protein(s)” refers to the recovery of all or part of membrane-protein(s) contained in a biological sample. Extracting said membrane-protein(s) can comprise in particular solubilizing and then isolating said membrane-protein(s) from the rest of the biological sample. Advantageously, the compound of the invention used to extract membrane-proteins also stabilizes the membrane-protein(s) such that the extracted membrane-protein(s) can preserve its native structure.

In step (a), a compound of the invention (or a detergent composition of the invention) is contacted with a biological sample comprising membrane-protein(s).

The biological sample of step (a) may be from any source, such as a human, animal, vegetal, bacterial, algal, fungal, protozoal, archaeal or viral source. The biological sample may for instance be obtained from microbial fermentation, cellular cultures, biological tissues (e.g. muscular tissues, bone tissues, mucosa, corneum, skin, connective tissues, or neural tissues), and/or biological body fluids (e.g. blood, sputum, lymph fluid, cerebrospinal fluid, urine, serum, plasma, sweat, various aspirates). The biological sample can be obtained from cells which may be eukaryotic or prokaryotic. Examples of cells include, but are not limited to, chondrocytes, osteoblasts, fibroblasts, blood cells, plasmocytes, neurons, hepatocytes, enterocytes, or cancer cells. Alternatively, cells may be unicellular organisms such as bacteria (e.g. E. coli), virus, fungi, protozoa, algae or archaea.

The biological sample of step (a) can be prepared by any techniques known to the skilled artisan.

In a particular embodiment, the biological sample comprises cell membranes fragments. Such biological sample can for instance be prepared according to the following procedure:
i) providing cells comprising membrane-proteins;
ii) subjecting said cells to lysis, for instance by ultrasonication, so as to produce a cell lysate;
iii) subjecting the cell lysate to centrifugation, so as to produce a precipitate comprising cell debris, and a supernatant comprising cell membrane fragments and soluble and peripheral proteins;
iv) recovering said supernatant;
v) subjecting said supernatant to ultracentrifugation so as to produce a precipitate comprising cell membrane fragments and a supernatant comprising soluble and peripheral proteins; and
vi) recovering said precipitate comprising cell membrane fragments.

The centrifugation rate in step (iii) can be adjusted by the skilled artisan. For instance, such centrifugation rate can be 5000 and 10 000 rpm. The ultracentrifugation rate in step (v) can also be adjusted by the skilled artisan. For instance, such ultracentrifugation rate can be comprised between 20 000 and 60 000 rpm.

The biological sample and the compound of the invention (or the detergent composition of the invention) may be contacted in any suitable solvent or buffer, such as Tris buffer, phosphate-buffered saline solution, carbonate buffer solution, citrate buffer solution, monochloracetate buffer solution, acetate buffer solution, or borate buffer solution.

The concentration Cₐ of the compound of the invention in step (a) is defined herein as the molar amount of the compound of the invention per volume unit of reaction medium of step (a). Such concentration Cₐ is typically higher or equal to the critical micellar concentration (CMC). In a particular embodiment, said concentration Cₐ is 2, 3, 4, 5, 10, 15 or 20 times the CMC of the compound of the invention in the conditions of step (a). In another particular embodiment, said concentration is defined by the following equation (1):

$${C}_{a} ( in mM ) = CMC + y \qquad ( 1 )$$

wherein CMC is the critical micellar concentration of the compound of the invention (in mM) and y is comprised between 1 mM and 10 mM, preferably between 1 mM and 5 mM.

The critical micellar concentration of a compound of the invention may be determined by surface tension measurement using the Whilelmy plate technique, by isothermal titration calorimetry (ITC), or any suitable technique known to the skilled artisan.

The contacting step (a) is advantageously carried out at room temperature, preferably for 1 hour to 50 hours, more preferably for 10 hours to 30 hours. As used herein, the expression “room temperature” refers to a temperature comprised between 5° C. and 40° C., preferably between 15° C. and 30° C.

Step (b) of the method of the invention comprises recovering membrane-protein(s).

The membrane-proteins can be recovered by any classical techniques known to the skilled artisan.

In one embodiment, step (b) comprises subjecting the mixture obtained in step (a) to ultracentrifugation, at a rate that can be adjusted by the skilled artisan, for instance between 20 000 and 60 000 rpm. In this embodiment, the ultracentrifugation is advantageously carried out for 15 min to 3 hours, preferably for 30 min to 2 hours, at a temperature comprised between 2° C. and 15° C. The ultracentrifugation typically produces a supernatant comprising the compound of the invention and membrane-proteins, and a precipitate comprising membrane debris. The supernatant can be recovered by any suitable technique known to the skilled artisan (such as by filtration or by sampling using a pipette).

In another aspect, the compounds as disclosed herein do not comprise a linker Y. In such aspect, the polar moiety X and the perfluorinated group Z are therefore directly linked to each other. Such compounds can be represented by the following formula (I-0): X—(Y)_{y}—Z (I-0), in which X, Y and Z are such as defined herein and y is 0. Equivalently, compounds of formula (I-0) can be represented by the formula X—Z, in which X and Z are such as defined herein.

Accordingly, another object of the present invention is a compound of formula (I-0): X—(Y)_{y}—Z (I-0) in which y is 0; or equivalently, of formula X—Z, in which X and Z are such as defined herein, including all particular and preferred embodiments described herein. In particular, X may be a maltoside, a lactobionamide or a sulfobetaine moiety, preferably a maltoside or a lactobionamide moiety, more preferably a maltoside moiety. In particular, Z may be a perfluorinated (C₃-C₁₂)cycloalkyl, preferably a perfluorinated (C₅-C₇)cycloalkyl. More preferably Z is a perfluorinated cyclohexyl. In particular, Z may be a perfluorinated isoheptyl or a perfluorinated isopentyl. Another object of the invention is a detergent composition comprising at least one compound of formula (I-0) as defined herein (or equivalently, of formula X—Z as defined herein). A further object of the invention is a use of a compound of formula (I-0) as defined herein (or equivalently, of formula X—Z as defined herein) or said detergent composition as defined herein, as a detergent for extracting membrane-proteins or synthesizing membrane-proteins in acellular system. In particular, said membrane-protein may be Bacteriorhodopsin or FhuA. A further object of the invention is an in-vitro method for extracting a membrane-protein from a biological sample comprising the following steps: (a) contacting a compound formula (I-0) as defined herein (or equivalently, of formula X—Z as defined herein) or said detergent composition as defined herein with a biological sample comprising a membrane-protein; and (b) recovering a membrane-protein.

The invention will also be described in further detail in the following examples, which are not intended to limit the scope of this invention, as defined by the attached claims.

### EXAMPLES

### Example 1: Preparation of the Compounds of the Invention

The detergents 5α-5c were synthesized in four steps, as illustrated in Scheme 1. Compounds 2a, 2b and 2c were prepared starting from peracetylated maltose by glycosylation reaction with allyl alcohol, buten-1-yl alcohol and penten-1-yl alcohol, respectively. The double bonds of the obtained compounds were then subjected to free radical reaction with perfluorocyclohexyl iodide in the presence of 1 M BEt3 in hexane. The addition of the fluoroalkyl chain to the double bonds was confirmed by ¹H- and ¹³C-NMR, which showed the disappearance of the signals corresponding to the double bond and the formation of new signals of —CHI. The iodine group of compounds 3a, 3b and 3c was reduced under H2 gas and in the presence of Pd/C as catalyst. The obtained compounds 4a, 4b and 4c were then deprotected under Zemplen conditions, using a catalytic amount of MeONa in MeOH to obtain the desired detergents 5a, 5b and 5c. The crude detergents were purified by chromatography and freeze-dried to give the pure detergents.

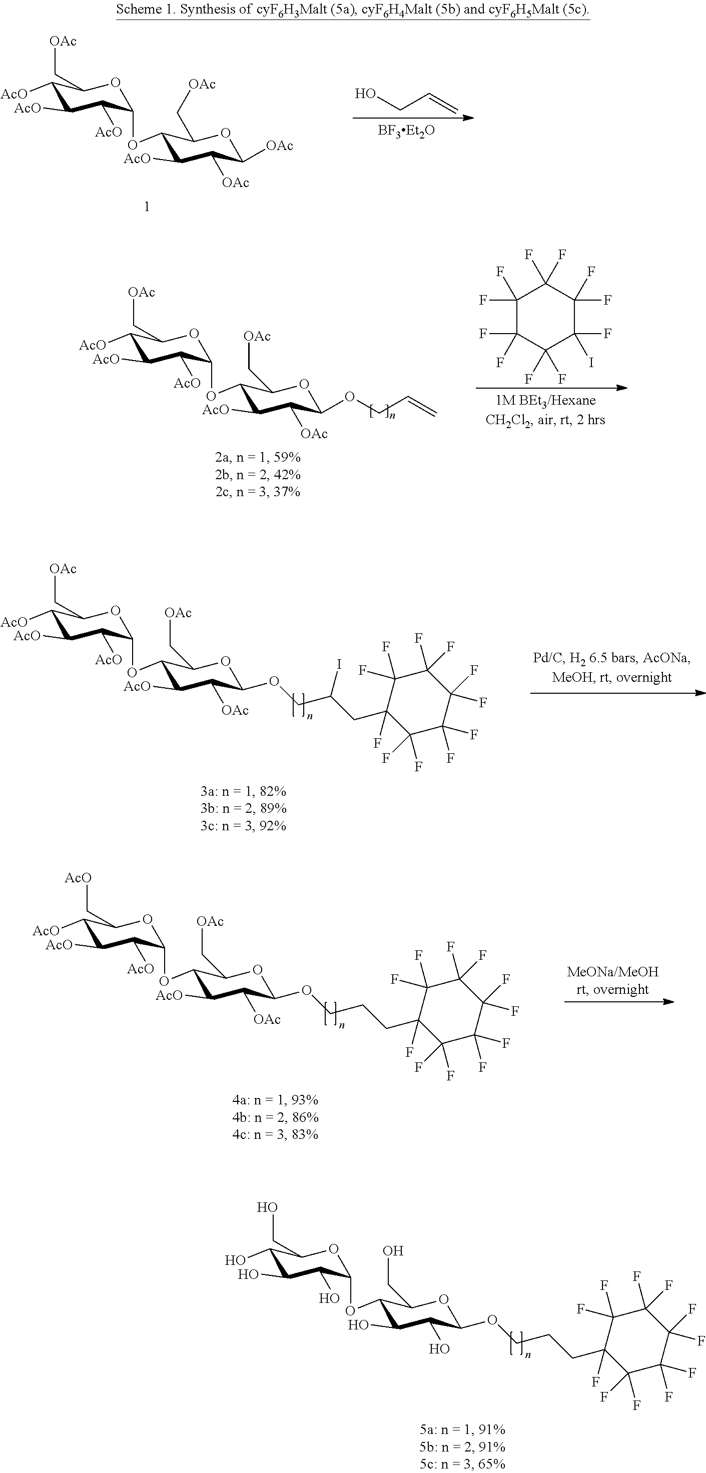

Prop-2-en-1-yl-2,3,6-tri-O-acetyl-4-O—(α-D-2′,3′,4′,6′-tetra-O-acety-glucopyranosy1)-β-D-glucopyranoside (2a). Under argon, octa-O-acetyl-ß-D-maltose (3.20 g, 4.71 mmol, 1.0 equiv) was dissolved in dry dichloromethane (10 mL) and the resulting solution was cooled down using an ice bath. Allyl alcohol (0.437 g, 7.54 mmol, 1.6 equiv) was first added followed by the dropwise addition of boron trifluoride diethyl ether complex (0.87 mL, 7.07 mmol, 1.5 equiv). The mixture was stirred at 0° C. for 2 h and kept at room temperature overnight. Dichloromethane (20 mL) was added, then the mixture was washed with saturated NaHCO₃ (2×20 mL) and brine (2×20 mL). The organic phase was collected and dried over anhydrous Na2SO₄, filtered, and the solvent was removed under reduced pressure. The crude compound was purified by column chromatography on silica gel (cyclohexane/ethyl acetate, 4:6, v/v) to get 2a (1.88 g, 59%) as a white powder. ¹H NMR (CDCl₃, 400 MHz): δ/ppm 5.89-5.78 (m, 1H), 5.40 (d, J=3.9 Hz, 1H), 5.35 (t, J=9.4 Hz, 1H), 5.28-5.17 (m, 3H), 5.04 (t, J=9.7, 1H), 4.87-4.82 (m, 2H), 4.57 (d, J=7.9 Hz, 1H), 4.47 (m, 1H), 4.33-4.19 (m, 3H), 4.11-3.93 (m, 4H), 3.67 (m, 1H), 2.14 (s, 3H), 2.09 (s, 3H), 2.03 (s, 3H), 2.01 (s, 6H), 1.99 (s, H). ¹³C NMR (CDCl₃, 100 MHz): δ/ppm 170.5, 170.5, 170.2, 169.9, 169.6, 169.4, 133.3, 117.7, 99.0, 95.5, 75.4, 72.7, 72.2, 72.1, 70.0, 69.3, 68.5, 68.0, 62.8, 61.5, 20.9, 20.8, 20.7, 20.6, 20.6, 20.6. IRMS (ESI+) m/z: [M+Na]⁺ calculated for C₂₉H₄₀O₁₈Na: 699.2112, found 699.2122.

But-3-en-1-yl-2,3,6-tri-O-acetyl-4-O—(α-D-2′″,3′″,4′″,6′-tetra-O-acetyl-glucopyranosyl)-β-D-glucopyranoside (2b). 2b was synthesized following the same procedure as for 2a, from octa-O-acetyl-ß-D-maltose (5.64 g, 8.31 mmol, 1.0 equiv), butyl alcohol (0.96 g, 13.30 mmol, 1.6 equiv), and boron trifluoride diethyl ether complex (1.5 mL, 12.47 mmol, 1.5 equiv). After purification by column chromatography on silica gel (cyclohexane/ethyl acetate, 8:2, v/v) compound 2b (2.39 g, 42%) was obtained as a white foam. ¹H NMR (CDCl₃, 400 MHz): S/ppm 5.76 (m, 1H). 5.40 (d, J=4.1 Hz, 1H), 5.35 (t, J=9.8 Hz, 1H), 5.24 (t, J=9.1 Hz, 1H), 5.09-5.01 (m, 3H), 4.87-4.79 (m, 2H), 4.53 (d, J=7.8, 1H), 4.46 (dd, J=2.6 Hz, J=12.3 Hz, 1H), 4.24 (m, 2H), 4.05-3.93 (m, 3H), 3.89 (m, 1H), 3.67 (m, 1H), 3.53 (m, 1H), 2.31 (m, 2H), 2.14 (s, 3H), 2.10 (s, 3H), 2.04 (s, 3H), 2.02 (s, 3H), 2.00 (s, 3H), 1.99 (s, 6H). ¹³C NMR (CDCl₃, 100 MHz): δ/ppm 170.7, 170.6, 170.4, 170.1, 169.7, 169.5, 134.6, 116.9, 100.4, 95.7, 75.5, 72.9, 72.3, 70.1, 69.5, 69.4, 68.6, 68.2, 63.0, 61.7, 33.9, 21.0, 21.0, 20.8, 20.8, 20.7, 20.7. HIRMS (ESI+) m/z: [M+NH₄]⁺ calculated for C₃₀H₄₆NO₁₈: 708.2709, found 708.2714.

Pent-4-en-1-yl-2,3,6-tri-O-acetyl-4-O—(α-D-2′″,3′″,4′″,6′-tetra-O-acetyl-glucopyranosyl)-β-D-glucopyranoside (2c). 2c was synthesized following the same procedure as for 2a, from octa-O-acetyl-ß-D-maltose (3.0 g, 4.42 mmol, 1.0 equiv), pentyl alcohol (0.571 g, 6.63 mmol, 1.5 equiv), and boron trifluoride diethyl ether complex (0.82 mL, 6.63 mmol, 1.5 equiv). After purification by column chromatography on silica gel (cyclohexane/ethyl acetate, 3:7, v/v) compound 2c (1.16 g, 37%) was obtained as a white foam. ¹H NMR (CDCl₃, 400 MHz): S/ppm 5.76 (m, 1H). 5.40 (m, 1H), 5.35 (t, J=9.6 Hz, 1H), 5.25 (t, J=9.0 Hz, 1H), 5.09-4.93 (m, 3H), 4.87-4.79 (m, 2H), 4.50 (d, J=7.9, 1H), 4.46 (dd, J=2.6 Hz, J=12.1 Hz, 1H), 4.25 (m, 2H), 4.05-3.93 (m, 3H), 3.85 (m, 1H), 3.66 (m, 1H), 3.48 (m, 1H), 2.13 (s, 3H), 2.09 (s, 3H), 2.06 (m, 2H), 2.04 (s, 3H), 2.01 (s, 6H), 1.99 (s, 6H), 1.62 (m, 2H). ¹³C NMR (CDCl₃, 100 MHz): δ/ppm 170.5, 170.5, 170.3, 170.0, 169.6, 169.4, 137.8, 115.1, 100.3, 95.5, 75.5, 72.8, 72.2, 72.0, 70.0, 69.3, 69.3, 68.5, 68.0, 62.9, 61.5, 29.8, 28.5, 26.9, 20.9, 20.8, 20.7, 20.6, 20.6, 20.5. IRMS (ESI+) m/z: [M+H]⁺ calculated for C₃₁H₄₅O₁₈: 705.2605, found 705.2600. IRMS (ESI+) m/z: [M+Na]⁺ calculated for C₃₁H₄₄NaO₁₈: 727.2420, found 727.2393.

2-iodo-3-(perfluorocyclohexyl)-propoxy-2,3,6-tri-O-acetyl-4-O—(α-D-2′,3′,4′,6′-tetra-O-acetyl-glucopyranosyl)-β-D-glucopyranoside (3a). To a solution of 2a (0.67 g, 0.99 mmol, 1.0 equiv) in dichloromethane (10 mL), perfluorocyclohexyl iodide (0.3 mL, 1.38 mmol, 1.4 equiv) and triethyl borane 1M in hexane (0.2 mL, 0.19 mmol, 0.2 equiv) were added. The mixture was flushed with air for 20 min and stirred at room temperature for 2 h. 50 mL of a diluted solution of Na2S₂O₃ was added and the aqueous solution was extracted with CH₂Cl₂ (2×50 mL). The organic fractions were collected, dried over anhydrous Na₂SO₄, filtered, and the solvent was removed under reduced pressure. The crude compound was purified by column chromatography on silica gel (cyclohexane/ethyl acetate, 3:7, v/v) to give compound 3a (0.82 g, 82%) as a white powder. ¹H NMR (CDCl₃, 400 MHz): S/ppm 5.41 (dd, J=1.1 Hz, J=4.0 Hz, 1H), 5.36 (td, J=0.9 Hz, J=10.0 Hz, 1H), 5.26 (td, J=2.5 Hz, J=9.1 Hz, 1H), 5.05 (td, J=2.2 Hz, J=9.9 Hz, 1H), 4.90-4.84 (m, 2H), 4.61 (d, J=7.7 Hz, 1H), 4.53-4.35 (m, 2H), 4.27-4.16 (m, 2H), 4.08 (m, 1H), 4.05-3.93 (m, 3H), 3.78 (m, 1H), 3.70 (m, 1H), 3.19 (m, 1H), 2.76 (m, 1H), 2.13* (s, 3H), 2.10* (s, 3H), 2.04-2.00 (m, 15H). ¹⁹F NMR (CDCl₃, 376 MHz): δ/ppm −118.0 (d, J=295 Hz, CF₂),-131.0 (dd, J=277 Hz, J=6285 Hz, 2×CF₂),-132.9 (dd, J=61 Hz, J=297 Hz, CF₂),-133.2 (dd, J=284 Hz, J=6783 Hz, CF₂),-185.8 (s, CF). ¹³C NMR (CDCl₃, 100 MHz): δ/ppm 170.5, 170.4, 170.2, 170.0, 169.5, 169.5, 169.4, 100.4, 99.6, 95.6, 75.2, 75.1, 74.9, 73.9, 72.6, 72.3, 71.9, 70.0, 69.3, 68.6, 68.0, 62.6, 61.5, 32.4, 20.9, 20.7, 20.7, 20.6, 20.6, 20.6, 20.5, 15.2, 14.8. HRMS (ESI+) m/z: [M+NH₄]⁺ calculated for C₃₅H₄₄F₁₁INO₁₈: 1102.1422, found 1102.1422.

3-iodo-4-(perfluorocyclohexyl)-butoxy-2,3,6-tri-O-acetyl-4-O—(α-D-2′,3′,4′,6′-tetra-O-acetyl-glucopyranosyl)-β-D-glucopyranoside (3b). Compound 3b was synthesized following the same procedure as for 3a, from 2b (1.39 g, 2.01 mmol, 1.0 equiv), perfluorocyclohexyl iodide (1.15 g, 2.81 mmol, 1.4 equiv) and triethyl borane 1M in hexane (0.4 mL, 0.4 mmol, 0.2 equiv). After purification by column chromatography on silica gel (cyclohexane/ethyl acetate, 2:8, v/v), compound 3b (1.96 g, 89%) was obtained as a white foam. ¹H NMR (CDCl₃, 400 MHz): δ/ppm 5.41 (d, J=4.0 Hz, 1H), 5.36 (t, J=10.0 Hz, 1H), 5.25 (q, J=9.3 Hz, 1H), 5.05 (t, J=10.0 Hz, 1H), 4.87-4.79 (m, 2H), 4.56-4.45 (m, 3H), 4.23 (m, 2H), 4.08-3.88 (m, 4H), 3.75-3.55 (m, 2H), 3.16-2.88 (m, 2H), 2.13* (s, 3H), 2.09* (s, 3H), 2.04* (s, 3H), 2.03-1.99 (m, 12H), 1.98-1.83 (m, 2H). ¹⁹F NMR (CDCl₃, 376 MHz): δ/ppm-117.9 (d, J=300 Hz, CF₂),-122.4 (dd, J=105 Hz, J=274 Hz, CF₂),-132.9 (dd, J=290 Hz, J=6800 Hz, CF₂),-132.7 (dd, J=300 Hz, J=764 Hz, CF₂),-139.0 (d, J=280 Hz, CF₂),-184.3 (s, CF). ¹³C-NMR (CDCl₃, 100 MHz): δ/ppm 170.6, 170.5, 170.4, 170.1, 169.6, 169.6, 169.5, 100.8, 100.1, 95.7, 75.6, 75.4, 72.8, 72.3, 72.2, 70.1, 69.6, 69.5, 68.2, 62.8, 61.6, 40.5, 37.1, 21.0, 20.9, 20.8, 20.7, 20.7, 20.6, 19.0, 18.4, 14.3. HRMS (ESI+) m/z: [M+NH₄]⁺ calculated for C₃₆H₄₆F₁₁INO₁₈: 1116.1578, found 1116.1576.

4-iodo-5-(perfluorocyclohexyl)-pentoxy-2,3,6-tri-O-acetyl-4-O—(α-D-2′,3′,4′,6′-tetra-O-acetyl-glucopyranosyl)-β-D-glucopyranoside (3c). Compound 3c was synthesized following the same procedure as for 3a, from 2c (1.19 g, 1.69 mmol, 1.0 equiv), perfluorocyclohexyl iodide (0.96 g, 2.37 mmol, 1.4 equiv) and triethyl borane 1M in hexane (0.3 mL, 0.3 mmol, 0.2 equiv). After purification by column chromatography on silica gel (cyclohexane/ethyl acetate, 2:8, v/v), compound 3b (1.74 g, 92%) was obtained as a white foam. ¹H NMR (CDCl₃, 400 MHz): δ/ppm 5.40 (d, J=3.9 Hz, 1H), 5.35 (t, J=10.0 Hz, 1H), 5.25 (t, J=9.2 Hz, 1H), 5.04 (t, J=10.0 Hz, 1H), 4.86-4.77 (m, 2H), 4.52-4.44 (m, 2H), 4.39 (m, 1H), 4.26-4.18 (m, 2H), 4.05-3.93 (m, 3H), 3.87 (m, 1H), 3.66 (m, 1H), 3.51 (m, 1H), 3.11-2.85 (m, 2H), 2.13 (s, 3H), 2.09 (s, 3H), 2.03 (s, 3H), 2.01 (s, 3H), 1.99 (2s, 9H), 1.83 (m, 2H), 1.66 (m, 2H). ¹⁹F NMR (CDCl₃, 376 MHz): δ/ppm-117.8 (d, J=297 Hz, CF₂),-122.4 (dd, J=125 Hz, J=280 Hz, CF₂),-132.9 (dd, J=288 Hz, J=6805 Hz, CF₂),-132.8 (dd, J=294 Hz, J=743 Hz, CF₂),-139.0 (d, J=287 Hz, CF₂),-184.8 (s, CF). ¹³C-NMR (CDCl₃, 100 MHz): δ/ppm 170.6, 170.5, 170.4, 170.1, 169.6, 169.5, 100.3, 95.7, 75.5, 72.9, 72.3, 72.2, 70.1, 69.5, 68.7, 68.6, 68.5, 68.2, 62.9, 61.6, 60.5, 40.5, 37.7, 37.6, 30.1, 22.0, 21.0, 20.9, 20.8, 20.7, 20.7, 14.3. HRMS (ESI+) m/z: [M+Na]⁺ calculated for C₃₇H₄₄F₁₁INaO₁₈:1135.1289, found 1135.1273.

3-(perfluorocyclohexyl)-propoxy-2,3,6-tri-O-acetyl-4-O—(α-D-2′,3′,4′,6′-tetra-O-acetyl-glucopyranosyl)-β-D-glucopyranoside (4a). Compound 3a (2.21 g, 2.04 mmol, 1.0 equiv) was dissolved in methanol, Pd/C (0.22 g, 10% w) and sodium acetate (0.53 g, 6.52 mmol, 3.2 equiv) were added portion-wise. The resulting solution was stirred under H₂(g) (6.5 bars) overnight. The resulting mixture was filtered over a pad of celite and the solvent was evaporated under reduced pressure. The crude product was dissolved in CH₂C₂ (50 mL) and washed with a diluted solution of Na₂S₂O₃ (50 mL). Then the aqueous phase was extracted with CH₂C₂ (2×50 mL). The organic fractions were collected, dried over anhydrous Na₂SO₄, filtered, and the solvent was removed under reduced pressure. The crude compound was purified by column chromatography on silica gel (cyclohexane/ethyl acetate, 3:7, v/v) to give compound 4a (2.05 g, 93%). ¹H NMR (CDCl₃, 400 MHz): δ/ppm 5.40 (d, J=3.9 Hz, 1H), 5.35 (t, J=9.9 Hz, 1H), 5.24 (t, J=9.0 Hz, 1H), 5.04 (t, J=10.0 Hz, 1H), 4.86-4.80 (m, 2H), 4.53 (d, J=8.0 Hz, 1H), 4.47 (dd, J=2.5 Hz, J=12.1 Hz, 1H), 4.26-4.18 (m, 2H), 4.05-3.87 (m, 4H), 3.67 (m, 1H), 3.57 (m, 1H), 2.30-2.19 (m, 2H), 2.12 (s, 3H), 2.09 (s, 3H), 2.03 (s, 3H), 2.01 (s, 3H), 1.99 (2s, 9H), 1.96-1.85 (m, 2H). ¹⁹F NMR (CDCl₃, 376 MHz): δ/ppm-118.3 (d, J=294 Hz, CF₂),-122.6 (d, J=280 Hz, CF₂),-133.5 (dd, J=286 Hz, J=6782 Hz, CF₂),-132.9 (dd, J=64 Hz, J=300 Hz, CF₂),-139.4 (d, J=285 Hz, CF₂),-185.9 (s, CF). ¹³C-NMR (CDCl₃, 100 MHz): δ/ppm 170.5, 170.4, 170.2, 170.0, 169.5, 169.4, 100.2, 95.6, 75.3, 72.6, 72.2, 72.0, 70.0, 69.3, 68.6, 68.5, 68.0, 62.7, 61.5, 60.5, 22.5, 22.3, 21.9, 20.9, 20.7, 20.6, 20.6, 20.5, 20.4. HRMS (ESI+) m/z: [M+NH₄]⁺ calculated for C₃₅H₄₅F₁₁NO₁₈: 976.2456, found 976.2457.

4-(perfluorocyclohexyl)-butoxy-2,3,6-tri-O-acetyl-4-O—(α-D-2′,3′,4′,6′-tetra-O-acetyl-glucopyranosyl)-β-D-glucopyranoside (4b). Compound 4b was synthesized following the same procedure as for 4a, from 3b (1.93 g, 1.76 mmol, 1.0 equiv), Pd/C (0.19 g, 10% w) and sodium acetate (0.46 g, 5.63 mmol, 3.2 equiv) under H₂(g) (6.5 bars) overnight. After purification by column chromatography on silica gel (cyclohexane/ethyl acetate, 2:8, v/v), compound 4b (1.48 g, 86%) was obtained as a white foam. ¹H NMR (CDCl₃, 400 MHz): δ/ppm 5.40 (d, J=4.1 Hz, 1H), 5.35 (t, J=10.0 Hz, 1H), 5.24 (t, J=9.1 Hz, 1H), 5.04 (t, J=10.0 Hz, 1H), 4.87-4.78 (m, 2H), 4.52 (d, J=8.0 Hz, 1H), 4.47 (dd, J=2.8 Hz, J=12.2 Hz, 1H), 4.26-4.18 (m, 2H), 4.05-3.93 (m, 3H), 3.89 (m, 1H), 3.67 (m, 1H), 3.51 (m, 1H), 2.22-2.13 (m, 2H), 2.12 (s, 3H), 2.09 (s, 3H), 2.03 (s, 3H), 2.01 (s, 3H), 1.99 (2s, 9H), 1.75-1.59 (m, 4H). ¹⁹F NMR (CDCl₃, 376 MHz): δ/ppm-118.3 (d, J=295 Hz, CF₂),-122.7 (d, J=285 Hz, CF₂),-132.8 (d, J=294 Hz, CF₂),-133.2 (dd, J=282 Hz, J=6794 Hz, CF₂),-139.4 (d, J=285 Hz, CF₂), −185.9 (s, CF). ¹³C-NMR (CDCl₃, 100 MHz): δ/ppm 170.5, 170.5, 170.4, 170.2, 169.9, 169.5, 169.4, 100.2, 95.6, 75.4, 72.8, 72.2, 72.1, 70.0, 69.3, 68.9, 68.5, 68.1, 62.8, 61.5, 29.4, 25.5, 25.3, 20.9, 20.7, 20.6, 20.5, 20.5, 20.4, 18.2. HRMS (ESI+) m/z: [M+NH₄]⁺ calculated for C₃₆H₄₇F₁₁NO₁₈: 990.2612, found 990.2615.

5-(perfluorocyclohexyl)-pentoxy-2,3,6-tri-O-acetyl-4-O—(α-D-2′,3′,4′,6′-tetra-O-acetyl-glucopyranosyl)-β-D-glucopyranoside (4c). Compound 4c was synthesized following the same procedure as for 4a, from 3c (1.69 g, 1.52 mmol, 1.0 equiv), Pd/C (0.17 g, 10% w) and sodium acetate (0.40 g, 4.86 mmol, 3.2 equiv) under H₂(g) (6.5 bars) overnight. After purification by column chromatography on silica gel (cyclohexane/ethyl acetate, 2:8, v/v), compound 4c (1.24 g, 83%) was obtained as a white foam. ¹H NMR (CDCl₃, 400 MHz): δ/ppm 5.40 (d, J=4.1 Hz, 1H), 5.35 (t, J=10.0 Hz, 1H), 5.24 (t, J=9.1 Hz, 1H), 5.04 (t, J=10.0 Hz, 1H), 4.87-4.78 (m, 2H), 4.52 (d, J=8.0 Hz, 1H), 4.47 (dd, J=2.8 Hz, J=12.2 Hz, 1H), 4.26-4.18 (m, 2H), 4.05-3.93 (m, 3H), 3.89 (m, 1H), 3.67 (m, 1H), 3.51 (m, 1H), 2.22-2.13 (m, 2H), 2.12 (s, 3H), 2.09 (s, 3H), 2.03 (s, 3H), 2.01 (s, 3H), 1.99 (2s, 9H), 1.75-1.59 (m, 4H). ¹⁹F NMR (CDCl₃, 376 MHz): δ/ppm-118.3 (d, J=297 Hz, CF₂),-122.7 (d, J=284 Hz, CF₂),-132.8 (d, J=300 Hz, CF₂),-133.2 (dd, J=286 Hz, J=6777 Hz, CF₂),-139.4 (d, J=285 Hz, CF₂), −185.8 (s, CF). ¹³C-NMR (CDCl₃, 100 MHz): δ/ppm 170.5, 170.5, 170.4, 170.2, 169.9, 169.5, 169.4, 100.2, 95.6, 75.4, 72.8, 72.2, 72.1, 70.0, 69.3, 68.9, 68.5, 68.1, 62.8, 61.5, 29.4, 25.5, 25.3, 20.9, 20.7, 20.6, 20.5, 20.5, 20.4, 18.2. HRMS (ESI+) m/z: [M+NH₄]⁺ calculated for C₃₇H₄₉F₁₁NO₁₈: 1004.2769, found 1004.2771.

3-(perfluorocyclohexyl)-propoxy-4-O—(α-D-glucopyranosyl)-β-D-glucopyranoside (5a). Compound 4a (1.3 g, 1.35 mmol, 1.0 equiv) was dissolved in methanol then a catalytic amount of sodium methoxide (30 mg, 0.54 mmol, 0.4 equiv) was added portion-wise. The resulting solution was stirred overnight at room temperature. The reaction mixture was neutralized by addition of Amberlite IRC-50 hydrogen form ion-exchange resin (2.0 g). The ion-exchange resin was filtered off and the solvent was removed under reduced pressure. The crude compound was purified by column chromatography on silica gel (CH₂Cl₂/CH₃OH, 85:15, v/v) to give compound 5a (0.82 g, 91%) as a white powder. ¹H NMR (CD₃OD, 400 MHz): δ/ppm 5.18 (d, J=3.4 Hz, 1H), 4.31 (d, J=7.8 Hz, 1H), 4.00 (m, 1H), 3.93-3.81 (m, 3H), 3.73-3.60 (m, 5H), 3.56 (t, J=9.2 Hz, 1H), 3.46 (dd, J=3.2 Hz, J=9.7 Hz, 1H), 3.39 (m, 1H), 3.28 (q, J=8.3 Hz, 2H), 2.49 (m, 2H), 1.98 (m, 2H). ¹⁹F NMR (CDCl₃, 376 MHz): δ/ppm-119.3 (d, J=300 Hz, CF₂),-123.9 (d, J=284 Hz, CF₂),-134.1 (d, J=297 Hz, CF₂),-134.6 (dd, J=291 Hz, J=6970 Hz, CF₂),-141.1 (d, J=285 Hz, CF₂),-186.8 (s, CF). ¹³C NMR (CD₃OD, 100 MHz): δ/ppm 102.8, 101.5, 79.9, 76.4, 75.2, 73.7, 73.4, 73.2, 72.8, 70.1, 68.1, 61.3, 60.8, 22.6, 22.4, 21.7. HRMS (ESI+) m/z: [M+H]⁺ calculated for C₂₁H₂₈F₁₁O₁₁: 665.1456, found 665.1457.

4-(perfluorocyclohexyl)-butoxy-4-O—(α-D-glucopyranosyl)-β-D-glucopyranoside (5b). Compound 5b was synthesized following the same procedure as for 5a, from 4b (1.44 g, 1.48 mmol, 1.0 equiv) and sodium methoxide (32 mg, 0.59 mmol, 0.4 equiv) overnight. After purification by column chromatography on silica gel (CH₂Cl₂/CH₃OH, 85:15, v/v), compound 5b (0.89 g, 91%) was obtained as a white powder. ¹H NMR (CD₃OD, 400 MHz): δ/ppm 5.16 (d, J=3.8 Hz, 1H), 4.28 (d, J=7.8 Hz, 1H), 3.97-3.86 (m, 2H), 3.85-3.76 (m, 2H), 3.71-3.51 (m, 5H), 3.52 (t, J=9.2 Hz, 1H), 3.44 (dd, J=3.8 Hz, J=9.6 Hz, 1H), 3.37 (m, 1H), 3.29-3.21 (m, 2H), 2.33 (m, 2H), 1.74 (m, 4H). ¹⁹F NMR (CDCl₃, 376 MHz): δ/ppm-119.3 (d, J=296 Hz, CF₂),-123.9 (d, J=282 Hz, CF₂),-134.1 (d, J=297 Hz, CF₂),-134.6 (dd, J=292 Hz,J=6980 Hz, CF₂),-141.1 (d, J=283 Hz, CF₂),-186.7 (s, CF). ¹³C NMR (CD₃OD, 100 MHz): δ/ppm 102.9, 101.5, 80.0, 76.5, 75.2, 73.7, 73.4, 73.3, 72.8, 70.1, 68.6, 61.3, 60.8, 29.3, 25.2, 25.0, 18.2. HRMS (ESI+) m/z: [M+H]⁺ calculated for C₂₂H₃₀F₁₁O₁₁: 679.1612, found 679.1620.

5-(perfluorocyclohexyl)-pentoxy-4-O—(α-D-glucopyranosyl)-β-D-glucopyranoside (5c). Compound 5c was synthesized following the same procedure as for 5a, from 4c (1.19 g, 1.21 mmol, 1.0 equiv) and sodium methoxide (26 mg, 0.48 mmol, 0.4 equiv) overnight. After purification by column chromatography on silica gel (CH₂Cl₂/CH₃OH, 95:5, v/v), compound 5c (0.55 g, 65%) was obtained as a white powder. ¹H NMR (CD₃OD, 400 MHz): δ/ppm 5.16 (d, J=3.8 Hz, 1H), 4.28 (d, J=7.8 Hz, 1H), 3.94-3.79 (m, 4H), 3.71-3.51 (m, 6H), 3.45 (dd, J=3.8 Hz, J=9.6 Hz, 1H), 3.38 (m, 1H), 3.29-3.21 (m, 2H), 2.34-2.24 (m, 2H), 1.72-1.65 (m, 4H), 1.55-1.48 (m, 2H). ¹⁹F NMR (CDCl₃, 376 MHz): δ/ppm-119.4 (d, J=297 Hz, CF₂),-123.9 (d, J=288 Hz, CF₂),-134.2 (d, J=297 Hz, CF₂),-134.6 (dd, J=284 Hz, J=6968 Hz, CF₂),-141.1 (d, J=282 Hz, CF₂),-186.6 (s, CF). ¹³C NMR (CD₃OD, 100 MHz): δ/ppm 102.9, 101.5, 80.0, 76.4, 75.2, 73.7, 73.4, 73.3, 72.8, 70.1, 69.0, 61.3, 60.8, 28.9, 25.8, 25.3, 25.1, 21.0. HRMS (ESI+) m/z: [M+H]⁺ calculated for C₂₃H₃₂F₁₁O₁₁: 693.1769, found 693.1778.

Compounds 8α-8c were synthesized using the same pathway. The double bonds of compounds 2a and 2c (preparation reported above) were subjected to free radical reaction with perfluoroisopentyl iodide for 6a and 6c or perfluoroisoheptyl iodide for 6b, in the presence of 1 M BEt3 in hexane. The iodine group of compounds was reduced under H₂ gas and in the presence of Pd/C as catalyst to lead to 7a, 7b and 7c. The obtained compounds were then deprotected under Zemplen conditions, using a catalytic amount of MeONa in MeOH to obtain the desired detergents 8a, 8b and 8c. The crude detergents were purified by chromatography and freeze-dried to give the pure detergents.

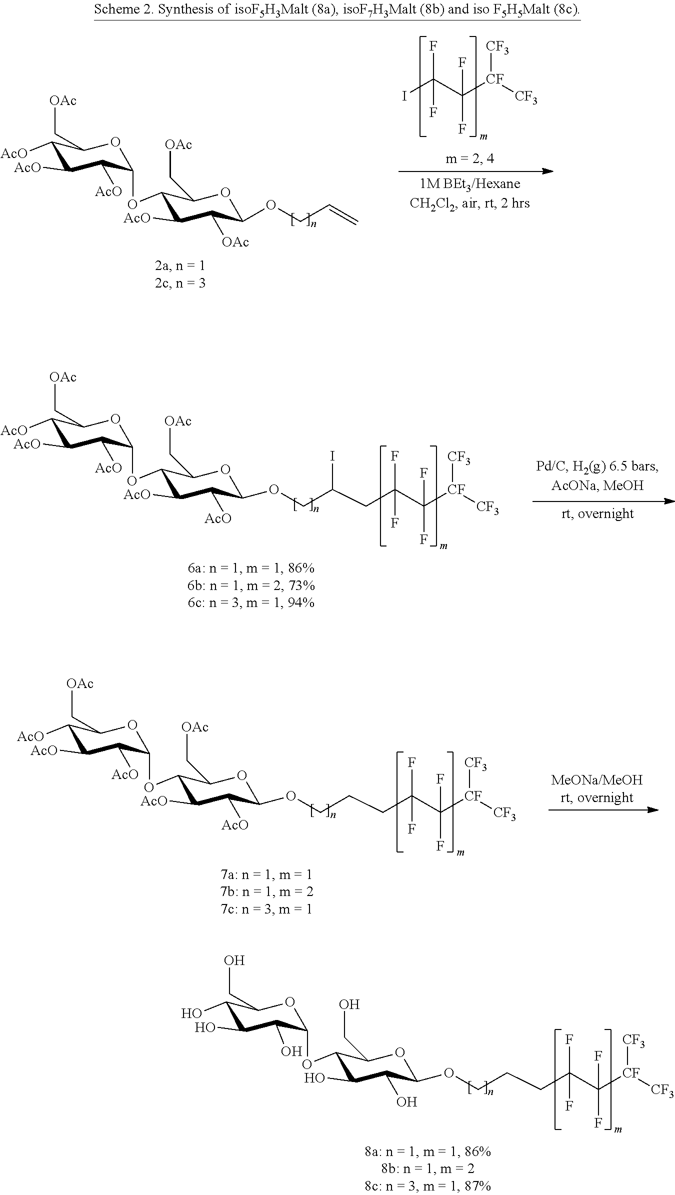

2-iodo-3-(perfluoroisopentyl)-propoxy-2,3,6-tri-O-acetyl-4-O—(α-D-2′,3′,4′,6′-tetra-O-acetyl-glucopyranosyl)-β-D-glucopyranoside (6a). To a solution of 2a (1.8 g, 2.66 mmol, 1.0 equiv) in dichloromethane (10 mL), perfluoroisopentyl iodide (0.7 mL, 3.59 mmol, 1.35 equiv) and triethyl borane 1M in hexane (0.5 mL, 0.53 mmol, 0.2 equiv) were added. The mixture was flushed with air for 20 min and stirred at room temperature for 2 h. 50 mL of a diluted solution of Na₂S₂O₃ was added and the aqueous solution was extracted with CH₂Cl₂ (2×50 mL). The organic fractions were collected, dried over anhydrous Na₂SO₄, filtered, and the solvent was removed under reduced pressure. The crude compound was purified by column chromatography on silica gel (cyclohexane/ethyl acetate, 8:2, v/v) to give compound 6a (2.47 g, 86%) as a beige foam. ¹H NMR (CDCl₃, 400 MHz): δ/ppm 5.40 (m, 1H), 5.35 (td, J=0.9 Hz, J=10 Hz, 1H), 5.25 (t, J=9.0 Hz, 1H), 5.04 (td, J=9.9 Hz, J=2.0 Hz, 1H), 4.88-4.83 (m, 2H), 4.61 (dd, J=7.7 Hz, J=2.1 Hz, 1H), 4.51-4.45 (m, 1H), 4.39-4.13 (m, 3H), 4.12-3.77 (m, 4H), 3.82-3.74 (m, 1H), 3.68 (m, 1H), 3.09-2.89 (m, 1H), 2.74-2.52 (m, 1H), 2.13* (s, 3H), 2.09* (s, 3H), 2.03* (s, 3H), 2.02-1.98 (m, 12H). ¹⁹F NMR (CDCl₃, 376 MHz): δ/ppm-71.9 (m, 2×CF₃),-113.3 (m, CF₂),-116.5 (m, CF₂),-185.8 (s, CF). ¹³C NMR (CDCl₃, 100 MHz): δ/ppm 170.5, 170.3, 170.2, 169.9, 169.5, 169.5, 169.4, 100.6, 99.7, 95.6, 75.1, 74.7, 73.7, 72.6, 72.3, 71.8, 70.0, 69.3, 68.6, 68.0, 62.6, 61.5, 37.6, 37.2, 26.9, 20.9, 20.7, 20.6, 20.6, 20.6, 20.6, 20.5, 13.7, 13.2. HRMS (ESI+) m/z: [M+H]⁺ calculated for C₃₄H₄₁F₁₁IO₁₈: 1073.1162, found 1073.1156.

2-iodo-3-(perfluoroisoheptyl)-propoxy-2,3,6-tri-O-acetyl-4-O—(α-D-2′,3′,4′,6′-tetra-O-acetyl-glucopyranosyl)-β-D-glucopyranoside (6b). Compound 6b was synthesized following the same procedure as for 6a, from 2a (1.58 g, 2.33 mmol, 1.0 equiv), perfluoroisoheptyl iodide (0.8 mL, 3.15 mmol, 1.35 equiv) and triethyl borane 1M in hexane (0.5 mL, 0.47 mmol, 0.2 equiv). After purification by column chromatography on silica gel (cyclohexane/ethyl acetate, 2:8, v/v), compound 6b (2.0 g, 73%) was obtained as a white foam. ¹H NMR (CDCl₃, 400 MHz): δ/ppm 5.40 (m, 1H), 5.34 (td, J=1.3 Hz, J=10 Hz, 1H), 5.25 (t, J=9.1 Hz, 1H), 5.04 (td, J=9.8 Hz, J=2.3 Hz, 1H), 4.88-4.82 (m, 2H), 4.61 (dd, J=7.8 Hz, J=2.1 Hz, 1H), 4.51-4.45 (m, 1H), 4.40-4.15 (m, 3H), 4.12-3.93 (m, 4H), 3.82-3.74 (m, 1H), 3.68 (m, 1H), 3.07-2.88 (m, 1H), 2.74-2.51 (m, 1H), 2.12* (s, 3H), 2.08* (s, 3H), 2.03 (s, 3H), 2.02-1.98 (m, 12H). ¹⁹F NMR (CDCl₃, 376 MHz): δ/ppm-71.8 (m, 2×CF₃),-113.5 (m, CF₂),-115.1 (s, CF₂),-120.6 (s, CF₂),-123.1 (m, CF₂),-186.0 (m, CF). ¹³C NMR (CDCl₃, 100 MHz): δ/ppm 170.5, 170.3, 170.2, 169.9, 169.5, 169.5, 169.4, 120.0, 117.1, 100.6, 99.7, 95.6, 75.1, 74.7, 73.7, 72.5, 72.3, 71.8, 70.0, 69.3, 68.6, 68.0, 62.6, 61.5, 37.6, 37.2, 26.9, 20.9, 20.7, 20.6, 20.6, 20.6, 20.5, 20.5, 13.7, 13.2. HRMS (ESI+) m/z: [M+NH₄]⁺ calculated for C₃₆H₄₄F₁₅INO₁₈: 1190.1358, found 1190.1355.

### 4-iodo-5-(perfluoroisopentyl)-propoxy-2,3,6-tri-O-acetyl-4-O—(α-D-2′,3′,4′,6′-tetra-O-acetyl-glucopyranosyl)-β-D-glucopyranoside (6c)

Compound 6c was synthesized following the same procedure as for 6a, from 2c (1.2 g, 1.74 mmol, 1.0 equiv), perfluoroisopentyl iodide (0.5 mL, 2.34 mmol, 1.35 equiv) and triethyl borane 1M in hexane (0.4 mL, 0.34 mmol, 0.2 equiv). After purification by column chromatography on silica gel (cyclohexane/ethyl acetate, 2:8, v/v), compound 6c (1.8 g, 94%) was obtained as a white foam. ¹H NMR (CDCl₃, 400 MHz): δ/ppm 5.40 (d, J=3.9 Hz, 1H), 5.35 (t, J=10.0 Hz, 1H), 5.25 (t, J=9.0 Hz, 1H), 5.05 (t, J=10.0 Hz, 1H), 4.87-4.78 (m, 2H), 4.53-4.47 (m, 2H), 4.34-4.18 (m, 3H), 4.05-3.94 (m, 3H), 3.91-3.85 (m, 1H), 3.67 (m, 1H), 3.53 (m, 1H), 2.98-2.69 (m, 2H), 2.13 (s, 3H), 2.09 (s, 3H), 2.04 (s, 3H), 2.02 (s, 3H), 2.00 (s, 3H), 2.00 (s, 6H), 1.85 (m, 2H), 1.72-1.57 (m, 2H). ¹⁹F NMR (CDCl₃, 376 MHz): δ/ppm-71.9 (m, 2×CF₃),-112.9 (m, CF₂),-116.4 (m, CF₂),-185.8 (s, CF). ¹³C NMR (CDCl₃, 100 MHz): δ/ppm 170.7, 170.5, 170.4, 170.1, 169.7, 169.5, 100.3, 95.7, 75.5, 72.9, 72.3, 70.1, 69.5, 68.6, 68.2, 62.9, 61.6, 41.8, 37.0, 30.0, 21.0, 20.9, 20.8, 20.7, 20.7, 20.2, 20.0. IRMS (ESI+) m/z: [M+H]* calculated for C₃₆H₄₅F₁₁IO₈: 1101.1475, found 1101.1476.

3-(perfluoroisopentyl)-propoxy-2,3,6-tri-O-acetyl-4-O—(α-D-2′,3′,4′,6′-tetra-O-acetyl-glucopyranosyl)-β-D-glucopyranoside (7a). Compound 6a (2.4 g, 2.24 mmol, 1.0 equiv) was dissolved in methanol, Pd/C (0.15 g, 50 mg/mmol) and sodium acetate (0.58 g, 7.16 mmol, 3.2 equiv) were added portion-wise. The resulting solution was stirred under H₂(g) (6.5 bars) overnight. The resulting mixture was filtered over a pad of celite and the solvent was evaporated under reduced pressure. The crude product was dissolved in CH₂C₂ (50 mL) and washed with a diluted solution of Na₂S₂O₃ (50 mL). Then the aqueous phase was extracted with CH₂C₂ (2×50 mL). The organic fractions were collected, dried over anhydrous Na₂SO₄, filtered, and the solvent was removed under reduced pressure. The crude compound was used in the next step without any further purification (crude compound 7a, 2.14 g). ¹H NMR (CDCl₃, 400 MHz): δ/ppm 5.40 (d, J=4.0 Hz, 1H), 5.35 (t, J=10.0 Hz, 1H), 5.24 (t, J=9.1 Hz, 1H), 5.05 (t, J=9.9 Hz, 1H), 4.87-4.79 (m, 2H), 4.52 (d, J=7.9 Hz, 1H), 4.48 (dd, J=2.7 Hz, J=12.1 Hz, 1H), 4.26-4.18 (m, 2H), 4.06-3.88 (m, 4H), 3.67 (m, 1H), 3.58 (m, 1H), 2.21-2.14 (m, 2H), 2.12 (s, 3H), 2.09 (s, 3H), 2.03 (s, 3H), 2.02 (s, 3H), 1.99 (3s, 9H), 1.93-1.83 (m, 2H). ¹⁹F NMR (CDCl₃, 376 MHz): δ/ppm-72.0 (m, 2×CF₃),-114.3 (m, CF₂),-116.3 (m, CF₂),-185.8 (m, CF). ¹³C-NMR (CDCl₃, 100 MHz): δ/ppm 170.7, 170.6, 170.4, 170.1, 169.7, 169.5, 100.3, 95.7, 75.5, 72.8, 72.3, 72.2, 70.1, 69.5, 68.7, 68.4, 68.1, 62.8, 61.6, 60.5, 27.6 (t, J=23 Hz), 21.0, 20.9, 20.8, 20.7, 20.7, 20.7, 20.5. IRMS (ESI+) m/z: [M+NH₄]⁺ calculated for C₃₄H₄₅F₁₁NO₁₈: 964.2456, found 964.2465.

3-(perfluoroisoheptyl)-butoxy-2,3,6-tri-O-acetyl-4-O—(α-D-2′,3′,4′,6′-tetra-O-acetyl-glucopyranosyl)-β-D-glucopyranoside (7b). Compound 7b was synthesized following the same procedure as for 7a, from 6b (2.0 g, 1.71 mmol, 1.0 equiv), Pd/C (0.05 g, 10% w) and sodium acetate (0.45 g, 5.46 mmol, 3.2 equiv) under H₂(g) (6.5 bars) overnight. The crude compound was used in the next step without any further purification (crude compound 7b, 1.56 g). ¹H NMR (CDCl₃, 400 MHz): δ/ppm 5.41 (d, J=4.0 Hz, 1H), 5.36 (t, J=10.0 Hz, 1H), 5.25 (t, J=9.1 Hz, 1H), 5.05 (t, J=9.8 Hz, 1H), 4.87-4.80 (m, 2H), 4.53 (d, J=8.0 Hz, 1H), 4.48 (dd, J=2.7 Hz, J=12.2 Hz, 1H), 4.27-4.19 (m, 2H), 4.06-3.89 (m, 4H), 3.67 (m, 1H), 3.58 (m, 1H), 2.20-2.13 (m, 2H), 2.13 (s, 3H), 2.10 (s, 3H), 2.04 (s, 3H), 2.02 (s, 3H), 1.99 (3s, 9H), 1.92-1.84 (m, 2H). ¹⁹F NMR (CDCl₃, 376 MHz): δ/ppm-71.8 (m, 2×CF₃),-114.4 (m, CF₂), −115.1 (m, CF₂),-120.7 (m, CF₂),-123.0 (m, CF₂),-186.0 (m, CF). ¹³C NMR (CDCl₃, 100 MHz): δ/ppm 170.7, 170.7, 170.5, 170.4, 170.1, 169.7, 169.5, 100.4, 95.7, 75.5, 72.8, 72.4, 72.2, 70.1, 69.5, 68.7, 68.4, 68.2, 62.9, 61.6, 27.7 (t, J=22.2 Hz), 21.0, 20.9, 20.8, 20.7, 20.7, 20.7, 20.5. HRMS (ESI+) m/z: [M+NH₄]⁺ calculated for C₃₆H₄₅F₁₅NO₁₈: 1064.2392, found 1064.2395.

5-(perfluoroisopentyl)-butoxy-2,3,6-tri-O-acetyl-4-O—(α-D-2′,3′,4′,6′-tetra-O-acetyl-glucopyranosyl)-β-D-glucopyranoside (7c). Compound 7c was synthesized following the same procedure as for 7a, from 6c (1.79 g, 1.63 mmol, 1.0 equiv), Pd/C (0.05 g, 10% w) and sodium acetate (0.43 g, 5.23 mmol, 3.2 equiv) under H₂(g) (6.5 bars) overnight. The crude compound was used in the next step without any further purification (crude compound 7c, 1.53 g). ¹H NMR (CDCl₃, 400 MHz): δ/ppm 5.41 (d, J=3.9 Hz, 1H), 5.36 (t, J=10.0 Hz, 1H), 5.25 (t, J=9.1 Hz, 1H), 5.05 (t, J=9.8 Hz, 1H), 4.87-4.79 (m, 2H), 4.52-4.46 (m, 2H), 4.27-4.20 (m, 2H), 4.06-3.94 (m, 3H), 3.85 (m, 1H), 3.67 (m, 1H), 3.48 (m, 1H), 2.13 (s, 3H), 2.10 (s, 3H), 2.04 (m, 5H), 2.02 (s, 3H), 2.00 (3s, 9H), 1.64-1.56 (m, 4H), 1.47-1.36 (m, 2H). ¹⁹F NMR (CDCl₃, 376 MHz): δ/ppm-71.9 (m, 2×CF₃),-114.3 (m, CF₂),-116.4 (m, CF₂),-185.8 (m, CF). ¹³C NMR (CDCl₃, 100 MHz): δ/ppm 170.7, 170.6, 170.4, 170.1, 169.7, 169.5, 100.4, 95.7, 75.6, 72.9, 72.3, 72.3, 70.1, 69.6, 69.5, 68.6, 68.2, 63.0, 61.7, 30.9 (t, J=22.7 Hz), 29.2, 25.6, 21.0, 20.9, 20.8, 20.7, 20.7, 20.0.

3-(perfluoroisopentyl)-propoxy-4-O—(α-D-glucopyranosyl)-β-D-glucopyranoside (8a). Compound 7a (2.14 g, 2.26 mmol, 1.0 equiv) was dissolved in methanol then a catalytic amount of sodium methoxide (48 mg, 0.90 mmol, 0.4 equiv) was added portion-wise. The resulting solution was stirred overnight at room temperature. The reaction mixture was neutralized by addition of Amberlite IRC-50 hydrogen form ion-exchange resin (2.0 g). The ion-exchange resin was filtered off and the solvent was removed under reduced pressure. The crude compound was purified by column chromatography on silica gel (CH₂Cl₂/CH₃OH, 95:5, v/v) to give compound 8a (1.27 g, 86%) as a white powder. ¹H NMR (CD₃OD, 400 MHz): δ/ppm 5.16 (d, J=3.8 Hz, 1H), 4.29 (d, J=7.8 Hz, 1H), 3.98 (m, 1H), 3.91-3.79 (m, 3H), 3.71-3.58 (m, 5H), 3.53 (t, J=9.1 Hz, 1H), 3.44 (dd, J=3.7 Hz, J=9.7 Hz, 1H), 3.37 (m, 1H), 3.25 (m, 2H), 2.35 (m, 2H), 1.90 (m, 2H). ¹⁹F NMR (CD₃OD, 376 MHz): δ/ppm-73.4 (m, 2×CF₃),-115.3 (m, CF₂),-117.1 (m, CF₂),-186.8 (m, CF). ¹³C NMR (CD₃OD, 100 MHz): δ/ppm 104.2, 102.9, 81.3, 77.8, 76.6, 75.1, 74.8, 74.6, 74.2, 71.5, 69.2, 62.8, 62.2, 28.9 (t, J=22.4 Hz), 21.9. HRMS (ESI+) m/z: [M+H]⁺ calculated for C₂₀H₂₈F₁₁O₁₁: 653.1456, found 653.1468.

3-(perfluoroisoheptyl)-propoxy-4-O—(α-D-glucopyranosyl)-β-D-glucopyranoside (8b). Compound 8b was synthesized following the same procedure as for 8a, from 7b (1.56 g, 1.58 mmol, 1.0 equiv) and sodium methoxide (34 mg, 0.63 mmol, 0.4 equiv) overnight. After purification by column chromatography on silica gel (CH₂Cl₂/CH₃OH, 85:15, v/v), compound 5b (1.01 g, 85%) was obtained as a white powder. ¹H NMR (CD₃OD, 400 MHz): δ/ppm 5.17 (d, J=3.8 Hz, 1H), 4.29 (d, J=7.7 Hz, 1H), 3.98 (m, 1H), 3.91-3.79 (m, 3H), 3.71-3.59 (m, 5H), 3.55 (t, J=9.2 Hz, 1H), 3.44 (dd, J=3.7 Hz, J=9.7 Hz, 1H), 3.40-3.36 (m, 1H), 3.25 (m, 2H), 2.34 (m, 2H), 1.91 (m, 2H). ¹⁹F NMR (CD₃OD, 376 MHz): δ/ppm-73.3 (m, 2×CF₃),-115.4 (m, CF₂),-116.0 (m, CF₂),-121.6 (m, CF₂),-124.0 (m, CF₂),-187.0 (m, CF). ¹³C NMR (CD₃OD, 100 MHz): δ/ppm 104.2, 102.9, 81.3, 77.8, 76.6, 75.1, 74.8, 74.6, 74.2, 71.5, 69.2, 62.8, 62.2, 28.9 (t, J=22.4 Hz), 21.9. HRMS (ESI+) m/z: [M+H]⁺ calculated for C₂₂H₂₈F₁₅O₁₁: 753.1392, found 753.1384.

5-(perfluoroisopentyl)-pentoxy-4-O—(α-D-glucopyranosyl)-β-D-glucopyranoside (8c). Compound 8c was synthesized following the same procedure as for 8a, from 7c (1.4 g, 1.44 mmol, 1.0 equiv) and sodium methoxide (31 mg, 0.57 mmol, 0.4 equiv) overnight. After purification by column chromatography on silica gel (CH₂Cl₂/CH₃OH, 85:15, v/v), compound 8c (0.85 g, 87%) was obtained as a white powder. ¹H NMR (CD₃OD, 400 MHz): δ/ppm 5.16 (d, J=3.7 Hz, 1H), 4.27 (d, J=7.8 Hz, 1H), 3.94-3.86 (m, 2H), 3.84-3.79 (m, 2H), 3.71-3.51 (m, 6H), 3.44 (dd, J=3.7 Hz, J=9.5 Hz, 1H), 3.39-3.35 (m, 1H), 3.24 (m, 2H), 2.17 (m, 2H), 1.65 (m, 4H), 1.52 (m, 2H). ¹⁹F NMR (CD₃OD, 376 MHz): δ/ppm-73.4 (m, 2×CF₃),-115.2 (m, CF₂),-117.2 (m, CF₂),-186.8 (m, CF). ¹³C NMR (CD₃OD, 100 MHz): δ/ppm 104.3, 102.9, 81.3, 77.9, 76.6, 75.1, 74.8, 74.7, 74.2, 71.5, 70.4, 62.7, 62.2, 31.7 (t, J=22.4 Hz), 30.4, 26.6, 21.1. HRMS (ESI+) m/z: [M+H]⁺ calculated for C₂₂H₃₂F₁₁O₁₁: 681.1769, found 681.1769.

Synthesis. Compound 12 (cyF₆H₃Lac) was synthesized in four steps, as illustrated in Scheme 3. The synthetic approach is very similar to the one used for the preparation of 5α-5c. Compound 9 was prepared from lactobionic acid by introduction of allylamine leading to the lactobionamide bond. The alcohol functions were next protected via peracetylation to give 9. The double bond of the obtained compound was then subjected to free radical reaction with perfluorocyclohexyl iodide in the presence of 1 M BEt3 in hexane. The addition of the fluoroalkyl moiety to the double bond was confirmed by ¹H- and ¹³C-NMR, which showed the disappearance of the signals corresponding to the double bond and the formation of new signals of —CHI. The iodine group of compound 10 was reduced under H₂ gas and in the presence of Pd/C as a catalyst. The obtained compounds 11 was then deprotected under Zemplen conditions, using a catalytic amount of MeONa in MeOH to obtain the desired detergent 12. The crude product was purified by chromatography and freeze-dried to give the pure detergent.

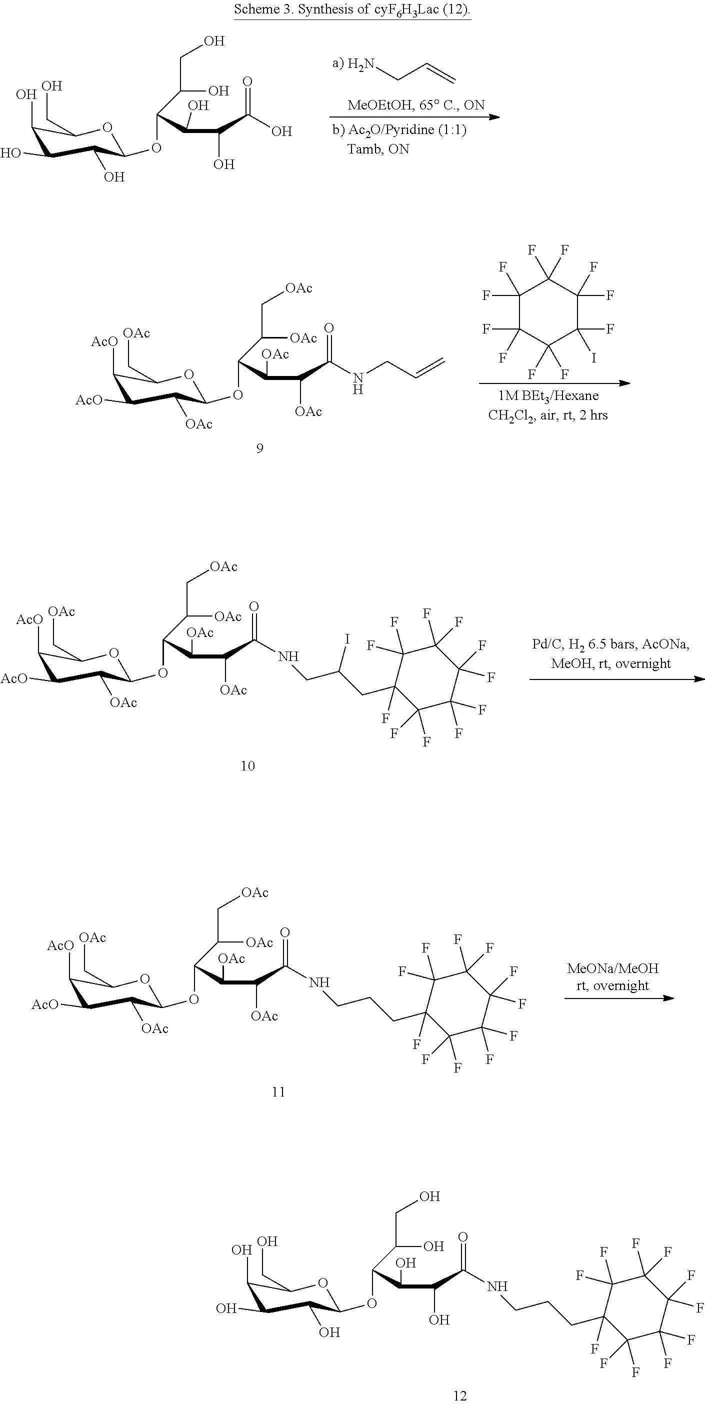

### Synthesis of Octaacetate-N-allyl-lactobionamide (9, AllyLacAc₈)

Lactobionic acid (10.0 g, 27.91 mmol, 1.0 equiv.) was put in suspension in 2-methoxyethanol (350 mL) at room temperature under stirring. Then, allylamine (12.5 mL, 166.6 mmol, 6.0 equiv.) was slowly added drop wise. The mixture was heated at 65° C. and stirred overnight. The solvent was removed under reduced pressure. The crude solid was washed in 350 mL of diethyl ether in a sonication bath and collected by filtration. Filtration through a short pad of silica gel (diam.=8 cm, h=10 cm, eluent=EtOAc/MeOH) led to N-allyllactobionamide (9.33 g, 84%) as a light orange solid which was directly used in the next step. At 0° C., N-allyllactobionamide was dissolved a 1:1 mixture pvridine/acetic anhydrid (100 mL. v/v) The mixture was stirred overnight at room temperature then poured into ice water. DCM (300 mL) was added followed by 10 min. of stirring at room temperature. The aqueous layer was extracted with DCM (2×50 mL). The combined organic phase was washed with a 1.0 M HCl solution until the aqueous layer reached pH≈1. The organic phase was then washed with a 10% wt NaHCO₃ solution, dried over MgSO₄, filtrated off and concentrated under reduced pressure. The crude compound 9 (15.54 g, 91%) was collected as a white solid, and used without further purification.

¹H NMR (400 MHz, CDCl₃), δ (ppm): 6.24 (1H, t, J=5.9 Hz), 5.79 (1H, m), 5.64 (1H, d, J=6.8 Hz), 5.53 (1H, dd, J=3.5 Hz, 6.7 Hz), 5.36 (1H, m), 5.20-5.10 (3H, m), 5.05 (1H, m), 4.98 (1H, dd, J=3.5 Hz, 10.4 Hz), 4.64 (1H, d, J=8.2 Hz), 4.50 (1H, dd, J=2.9 Hz, 12.2 Hz), 4.33 (1H, m), 4.12 (2H, m), 4.01 (1H, dd, J=5.4 Hz, 12.2 Hz), 3.95-3.85 (2H, m), 3.79 (1H, m), 2.15 (3H, s), 2.15 (3H, s), 2.10-2.00 (15H, m), 1.96 (3H, s). ¹³C NMR (100 MHz, CDCl₃), δ (ppm): 170.6, 170.5, 170.3, 170.2, 169.9, 169.8, 169.8, 169.4, 167.2, 133.5, 116.9, 101.9, 77.3, 71.6, 71.1, 69.8, 69.3, 69.1, 66.9, 61.8, 61.0, 41.8, 20.9, 20.9, 20.8, 20.8, 20.7, 20.7, 20.6. HRMS (ESI-TOF): [M+H]⁺ calculated for C₃₁H₄₄NO₁₉: 734.2508, found: 734.2516.

2-iodo-3-(perfluorocyclohexyl)-N-propane Octaacetate lactobionamide (10). To a solution of 9 (5 g, 6.82 mmol, 1.0 equiv) in dichloromethane (50 mL), perfluorocyclohexyl iodide (4.1 g, 10.22 mmol, 1.5 equiv) and triethyl borane 1M in hexane (1.4 mL, 1.36 mmol, 0.2 equiv) were added. The mixture was flushed with air for 20 min and stirred at room temperature for 2 h. 50 mL of a diluted solution of Na₂S₂O₃ was added and the aqueous solution was extracted with CH₂Cl₂ (2×50 mL). The organic fractions were collected, dried over anhydrous Na₂SO₄, filtered, and the solvent was removed under reduced pressure. The crude compound was purified by column chromatography on silica gel (cyclohexane/ethyl acetate, from 7:3 to 3:7, v/v) to give compound 10 (7.49 g, 96%) as a white powder.

¹H NMR (CDCl₃, 400 MHz): δ/ppm 6.63 (m, 1H), 5.65-5.53 (m, 2H), 5.38 (d, J=3.3 Hz, 1H), 5.21-5.17 (m, 1H), 5.10-5.05 (m, 1H), 5.02-4.99 (m, 1H), 4.64 (dd, J=8.0 Hz, J=10.1 Hz, 1H), 4.57-4.47 (m, 2H), 4.33-4.30 (m, 1H), 4.23-4.07 (m, 2H), 4.02 (m, 1H), 3.94-3.90 (m, 1H), 3.77-3.70 (m, 1H), 3.58-3.49 (m, 1H), 2.94 (m, 2H), 2.21 (s, 3H), 2.17 (s, 3H), 2.09 (s, 3H), 2.09 (s, 6H), 2.05-2.04 (m, 6H), 1.98 (s, 3H). ¹⁹F NMR (CDCl₃, 376 MHz): δ/ppm-117.9 (d, J=295 Hz, CF₂),-122.3 (d, J=275 Hz, CF₂),-132.7 (dd, J=284 Hz, J=550 Hz, CF₂),-132.8 (dd, J=284 Hz, J=6800 Hz, CF₂),-139.0 (dd, J=289 Hz, CF₂),-184.8 (d, J=97 Hz). ¹³C NMR (CDCl₃, 100 MHz): δ/ppm 170.7, 170.3, 170.2, 170.0, 169.9, 169.8, 169.5, 167.8, 101.8, 77.4, 71.7, 71.2, 71.1, 69.9, 69.1, 69.0, 66.9, 61.9, 61.0, 47.5, 34.0, 21.0, 20.9, 20.9, 20.8, 20.8, 20.7, 20.7, 20.4. HRMS (ESI+) m/z: [M+H]⁺ calculated for C₃₄H₄₄F₁₁INO₁₉: 1142.1371, found 1142.1367.

### 3-(perfluorocyclohexyl)-N-propane Octaacetate lactobionamide (11)

Compound 10 (3 g, 2.63 mmol, 1.0 equiv) was dissolved in methanol (60 mL), Pd/C (0.22 g, 50 mg/mmol) and sodium acetate (0.6 g, 8.41 mmol, 3.2 equiv) were added portion-wise. The resulting solution was stirred under H₂(g) (6.5 bars) overnight. The resulting mixture was filtered over a pad of celite and the solvent was evaporated under reduced pressure. The crude product was dissolved in CH₂Cl₂ (50 mL) and washed with a diluted solution of Na₂S₂O₃ (50 mL). Then the aqueous phase was extracted with CH₂C₂ (2×50 mL). The organic fractions were collected, dried over anhydrous Na₂SO₄, filtered, and the solvent was removed under reduced pressure. The crude compound was purified by column chromatography on silica gel (cyclohexane/ethyl acetate, from 7:3 to 0:1, v/v) to give compound 3 (1.55 g, 58%).

¹H NMR (CDCl₃, 400 MHz): δ/ppm 6.31 (t, J=5.9 Hz, 1H), 5.57-5.53 (m, 2H), 5.38 (dd, J=0.8 Hz, J=3.4 Hz, 1H), 5.17 (dd, J=7.9 Hz, J=10.4 Hz, 1H), 5.07 (m, 1H), 5.00 (dd, J=3.4 Hz, J=10.4 Hz, 1H), 4.64 (d, J=7.9 Hz, 1H), 4.53 (dd, J=2.6 Hz, J=12.4 Hz, 1H), 4.29 (dd, J=3.3 Hz, J=6.9 Hz, 1H), 4.19-4.06 (m, 2H), 4.00 (dd, J=5.4 Hz, J=12.3 Hz, 1H), 3.90 (t, J=6.7 Hz, 1H), 3.47-3.38 (m, 1H), 3.30-3.22 (m, 1H), 2.24-2.19 (m, 2H), 2.16 (2s, 6H), 2.08 (s, 3H), 2.07 (s, 3H), 2.06 (s, 3H), 2.04 (s, 3H), 2.03 (2s, 6H), 1.98 (s, 3H), 1.89-1.81 (m, 2H). ¹⁹F NMR (CDCl₃, 376 MHz): δ/ppm-118.2 (d, J=296 Hz),-131.0 (dd, J=286 Hz, J=6270 Hz),-132.8 (d, J=290 Hz),-133.1 (dd, J=290 Hz, J=6780 Hz),-185.5 (s). ¹³C-NMR (CDCl₃, 100 MHz): δ/ppm 170.7, 170.3, 170.2, 170.1, 169.9, 169.8, 169.5, 167.6, 101.8, 77.2, 71.7, 71.2, 71.1, 69.9, 69.3, 69.2, 66.9, 61.9, 61.0, 39.0, 23.3, 23.0, 22.0, 20.9, 20.9, 20.8, 20.8, 20.7, 20.6. HRMS (ESI+) m/z: [M+H]⁺ calculated for C₃₇H₄₅F₁₁N019: 1016.2410, found 1016.2411.

### 3-(perfluorocyclohexyl)-N-propane Octahydroxy lactobionamide (12)

Compound 11 (3.8 g, 3.74 mmol, 1.0 equiv) was dissolved in methanol then a catalytic amount of sodium methoxide (80 mg, 1.5 mmol, 0.4 equiv) was added portion-wise. The resulting solution was stirred overnight at room temperature. The reaction mixture was neutralized by addition of Amberlite IRC-50 hydrogen form ion-exchange resin (2.0 g). The ion-exchange resin was filtered off and the solvent was removed under reduced pressure. The crude compound was purified by permeation gel chromatography on LH₂O Sephadex (CH₃OH) to give compound 12 (2.45 g, 96%) as a white powder.

¹H NMR (DMSO-d6, 400 MHz): δ/ppm 7.87 (t, J=5.9 Hz, 1H), 5.26 (d, J=5.6 Hz, 1H), 5.16 (d, J=3.8 Hz, 1H), 4.79-4.76 (m, 1H), 4.62 (t, J=5.6 Hz, 1H), 4.51-4.46 (m, 2H), 4.26 (d, J=7.3 Hz, 1H), 4.11-4.07 (m, 2H), 4.01-3.98 (m, 1H), 3.95 (m, 1H), 3.72-3.67 (m, 2H), 3.63-3.56 (m, 2H), 3.53-3.23 (m, 8H), 3.18-3.13 (m, 2H), 2.41-2.24 (m, 2H), 1.74 (m, 2H). ¹⁹F NMR (DMSO-d6, 376 MHz): δ/ppm-117.8 (d, J=295 Hz),-130.6 (dd, J=280 Hz, J=6250 Hz), −132.3 (d, J=287 Hz),-132.7 (dd, J=280 Hz, J=6750 Hz),-185.2 (s). ¹³C NMR (DMSO-d6, 100 MHz): δ/ppm 172.6, 104.8, 101.5, 83.5, 75.6, 73.2, 71.9, 71.4, 71.1, 70.6, 68.0, 62.3, 60.3, 48.6, 37.6, 22.5, 22.3, 21.4. HRMS (ESI+) m/z: [M+H]⁺ calculated for C₂₁H₂₉F₁₁NO₁₁: 680.1565, found 680.1573.

Synthesis. Compound 17 (cyF₆H₅SB) was prepared from commercial pent-4-enol according the synthetic route described in Scheme 4. This approach involves the introduction of the fluorinated chain by the radical addition of perfluorocyclohexyl iodide on pent-4-enol with the use of triethylborane in the presence of oxygen to obtain 13 in 92% yield. Compound 13 was then submitted to reduction by a catalytic hydrogenation in the presence of Pd/C and AcONa to give 14 in 68% yield. The mesyl derivative 15 was prepared in 90% yield by treating the alcohol 14 with methanesulfonyl chloride in the presence of triethylamine. Then nucleophilic substitution of the mesyl group by dimethylamine gave 16. Finally, the reaction of 16 with propane-1,3-sultone yielded the sulfobetaine derivative cyF₆H₅SB as a white precipitate in 72% yield.

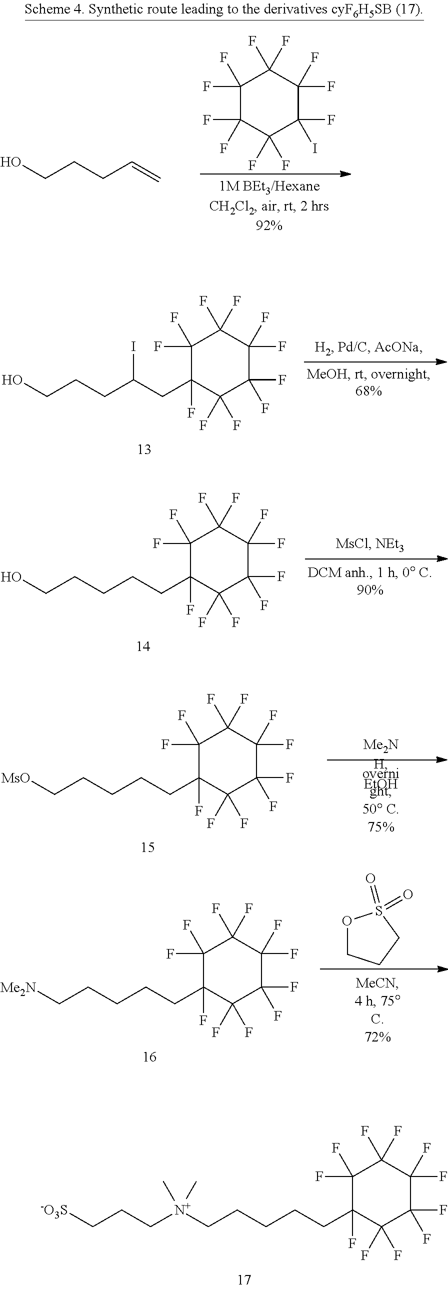

4-iodo-5-(perfluorocyclohexyl)-pentanol (13). To a solution of pent-4-en-1-ol (2.3 g, 26.7 mmol, 1.0 equiv) in CH₂Cl₂, perfluorocyclohexyliodide (12.53 g, 30.7 mmol, 1.15 equiv) and triethylborane 1M in hexane (0.78 mL, 0.78 mmol, 0.03 equiv) were added. The mixture was flushed with air and stirred at room temperature for 3 h. After completion of the reaction, a diluted solution of Na₂S₂O₃ was added and the aqueous layer was extracted with CH₂Cl₂ (3×). The organic fractions were collected, dried over anhydrous Na₂SO₄, filtered, and then concentrated under reduced pressure. The crude product was purified by flash chromatography (cyclohexane/AcOEt, 9:1 v/v) to yield 13 as a white solid (12.2 g, 92%). ¹H NMR (400 MHz, CDCl₃): δ 4.47 (m, 1H), 3.72 (t, J=6.2 Hz, 2H), 3.14-2.90 (m, 2H), 1.95-1.88 (m, 2H), 1.87-1.78 (m, 1H), 1.74-1.64 (m, 1H). ¹⁹F NMR (376 MHz, CDCl₃): δ −117.8 (d, J=295 Hz, CF₂), −122.4 (dd, J=108 Hz, J=287 Hz, CF₂),-132.9 (dd, J=295 Hz, J=730 Hz, CF₂),-132.6 (dd, J=285 Hz, J=6800 Hz, CF₂),-139.0 (d, J=285 Hz, CF₂),-184.9 (s, CF). ¹³C{¹H} NMR (100 MHz, CDCl₃): δ 61.8, 37.6, 36.9, 32.9, 22.4. IRMS (ESI+) m/z: [M−H₂O+H]⁺ calcd for C₁₀H₉F₁₁I, 476.9573; found, 476.9580.

5-(perfluorocyclohexyl)-pentanol (14). Compound 13 (3.5 g, 7.08 mmol, 1.0 equiv) was dissolved in MeOH and Pd/C (350 mg, catalytic amount, 10% mass) and sodium acetate (1.86 g, 22.7 mmol, 3.2 equiv) were added. The reaction mixture was stirred overnight under a 6.5 bar pressure of H₂ in a hydrogenation reactor and then filtered through a pad of Celite and concentrated under reduced pressure. A diluted solution of Na₂S₂O₃ was added to the crude product and extracted with CH₂Cl₂ (3×). The organic fractions were collected, dried over anhydrous Na₂SO₄, filtered, and concentrated under reduced pressure. The crude product was purified by flash chromatography (cyclohexane/AcOEt, 9:1 to 7:3) to yield 14 as a colorless oil (1.77 g, 68%). ¹H NMR (400 MHz, CDCl₃): δ 3.67 (t, J=6.4 Hz, 2H), 2.23-2.13 (m, 2H), 1.72-1.59 (m, 4H), 1.50-1.44 (m, 2H). ¹⁹F NMR (375 MHz, CDCl₃): δ −118.4 (d, J=295 Hz, CF₂), −122.2 (d, J=287 Hz, CF₂),-132.9 (d, J=293 Hz, CF₂),-132.8 (dd, J=287 Hz, J=6785 Hz, CF₂),-139.5 (d, J=285 Hz, CF₂),-184.9 (s, CF). ¹³C{¹H} NMR (100 MHz, CDCl₃): δ 62.6, 32.3, 26.2, 25.8, 21.3. HIRMS (ESI+) m/z: [M−H₂O+H]⁺ calcd for C₁₁H₁₀F₁₁, 351.0601; found, 351.0608.

5-(perfluorocyclohexyl)-pentanyl methanesulfonate (15). To a solution of 14 (2.0 g, 5.43 mmol, 1.0 equiv) in CH₂Cl₂, triethylamine (2.2 mL, 16.29 mmol, 3.0 equiv) was added. The reaction mixture was cooled down to 0° C. and MsCl (0.63 mL, 8.1 mmol, 1.5 equiv) was added dropwise. The mixture was stirred at 0° C. for 1 h and then poured into water and extracted with CH₂Cl₂ (3×). The organic fractions were collected and washed with 1M HCl, dried over anhydrous MgSO₄, filtered and then concentrated under reduced pressure. The crude product was purified by flash chromatography (cyclohexane/AcOEt, 9:1 to 8:2) to yield 15 as a colorless oil (2.19 g, 90%). ¹H NMR (400 MHz, CDCl₃): δ 4.25 (t, J=6.3 Hz, 2H), 3.01 (s, 3H), 2.24-2.15 (m, 2H), 1.85-1.78 (m, 2H), 1.74-1.66 (m, 2H), 1.56-1.48 (m, 2H). ¹⁹F NMR (375 MHz, CDCl₃): δ −118.4 (d, J=295 Hz, CF₂),-122.7 (d, J=285 Hz, CF₂),-132.8 (d, J=293 Hz, CF₂),-132.8 (dd, J=285 Hz, J=6780 Hz, CF₂),-139.4 (d, J=285 Hz, CF₂),-185.8 (s, CF). ¹³C{¹H} NMR (100 MHz, CDCl₃): δ 69.4, 37.6 28.9, 25.9, 25.7, 21.1. HIRMS (ESI+) m/z: [M+H]⁺ calcd for C₁₂H₁₄F₁₁O₃S, 447.0483; found, 447.0471.

5-(perfluorocyclohexyl)-N,N-dimethylpentan-1-amine (16). To a solution of dimethylamine (1.40 mL, 7.72 mmol, 3.0 equiv) in EtOH, compound 15 (1.15 g, 2.57 mmol, 1.0 equiv) in EtOH was added. The reaction mixture was stirred at 50° C. for 24 h then was poured into water. A solution of NaOH 1N was added before extraction with Et₂O (3×). The organic fractions were collected, dried over a hydrous MgSO₄, filtered and concentrated under reduced pressured to yield to crude 16 as an orange oil (0.77 g, 75%). ¹H NMR (400 MHz, CDCl₃): δ 2.38 (t, J=7.2 Hz, 2H), 2.32 (s, 6H), 2.22-2.13 (m, 2H), 1.71-1.63 (m, 2H), 1.61-1.55 (m, 2H), 1.45-1.38 (m, 2H). ¹⁹F NMR (375 MHz, CDCl₃): δ −118.3 (d, J=295 Hz, CF₂),-122.7 (d, J=285 Hz, CF₂),-132.8 (d, J=293 Hz, CF₂),-132.8 (dd, J=285 Hz, J=6795 Hz, CF₂),-139.4 (d, J=285 Hz, CF₂),-185.8 (s, CF). ¹³C{¹H} NMR (100 MHz, CDCl₃): δ 59.3, 45.2, 27.6, 26.9, 25.8, 21.4. HRMS (ESI+) m/z: [M+H]*calcd for C₁₃H₁₇F₁₁N, 396.1180; found, 396.1189.

### 3-(dimethyl(5-(perfluorocyclohexyl)pentyl)ammonio)propane-1-sulfonate (17)

Compound 16 (0.37 g, 0.94 mmol, 1.0 equiv) and 1,3-propanesultone (0.40 g, 3.28 mmol, 3.5 equiv) were dissolved in anhydrous acetonitrile. The reaction mixture was stirred at 75° C. for 6 h under an argon atmosphere. The mixture was concentrated under reduced pressure, then solubilized in MeOH and precipitated in Et₂O at 0° C. The obtained precipitate was filtered, solubilized again in hot MeOH, precipitated twice in cold AcOEt, rinsed with Et₂O and dried to yield 17 as a beige solid (0.35 g, 72%). ¹H NMR (400 MHz, CD₃OD): δ 3.52 (m, 2H), 3.36-3.34 (m, 2H), 3.11 (s, 6H), 2.88 (t, J=6.7, 2H), 2.41-2.32 (m, 2H), 2.24-2.17 (m, 2H), 1.91-1.83 (m, 2H), 1.83-1.74 (m, 2H), 1.53-1.45 (m, 2H). ¹⁹F NMR (375 MHz, CD₃OD): δ −119.3 (d, J=300 Hz, CF₂),-123.9 (d, J=285 Hz, CF₂),-134.2 (d, J=295 Hz, CF₂),-134.3 (dd, J=285 Hz, J=6990 Hz, CF₂),-141.2 (d, J=285 Hz, CF₂),-186.7 (s, CF). ¹³C{¹H} NMR (100 MHz, CD₃OD): δ 65.1, 63.8, 51.3, 48.2, 27.4, 26.4, 23.2, 22.3, 19.9. HRMS (ESI+) m/z: [M+H]⁺ calcd for C₁₆H₂₃F₁₁NO₃S, 518.1218; found, 518.1223.

SFT for CMC determination. The surface activity of the compounds of the invention in solution at the air/water interface was determined using a K100 tensiometer (Kruss, Hamburg, Germany). Surface tensions were determined by dilution of stock solutions (13 g/L for compound 5a (cyF₆H₃Malt), 4.6 g/L for compound 5b (cyF₆H₄Malt), 2 g/L for compound 5c (cyF₆H₅Malt), 10 g/L for compound 8a (isoF₅H₃Malt), 1.7 g/L for compound 8c (isoF₅H₅Malt) and 0.4 g/L for compound 8b (isoF₇H₃Malt), ˜4×CMC) using the Wilhelmy plate technique. In a typical experiment, 13 concentration steps were prepared from solutions equilibrated overnight before measurement. All measurements were performed at (20.0±0.5)° C. until standard deviation reach ±0.05 or during at least 30 min.

The CMC values are reported in the following table 1.

TABLE 1
Critical micellar concentration and surface tension at
the CMC for the detergents 5a-5c, 8a-8c, 12 and 17.

| | 5a | 5b | 5c | 8a | 8b | 8c | 12 | 17 |
|---|---|---|---|---|---|---|---|---|
| CMC | 3.47 ± | 2.29 ± | 0.70 ± | 2.66 ± | 0.11 ± | 0.53 ± | 6.32 ± | 3.20 ± |
| (mM) | 0.25 | 0.02 | 0.01 | 0.28 | 0.02 | 0.04 | 0.40 | 0.17 |
| γCMC | 23.9 ± | 22.7 ± | 23.16 ± | 22.5 ± | 17.4 ± | 22.1 ± | 24.4 ± | 23.7 ± |
| (mN/m) | 0.1 | 0.7 | 0.2 | 0.2 | 0.6 | 0.1 | 0.9 | 1.25 |

### Example 2: Solubilization of Large Unilamellar Vesicles

Preparation of lipid vesicles. To prepare LUVs, POPC in powder form was weighed on a high-precision XP Delta Range microbalance (Mettler Toledo, Greifensee, Switzerland) and suspended in phosphate buffer (10 mM Na₂HIPO₄/NaH₂PO₄, 150 mM NaCl, pH 7.4). The solution was vortexed for 15 min at room temperature and extruded in a LiposoFast extruder (Avestin, Mannheim, Germany) with at least 35 extrusion steps through two stacked polycarbonate membranes with a pore diameter of 100 nm (Avestin). The hydrodynamic diameter of the LUVs was distributed around 120-130 nm, as shown by DLS.

Kinetics of vesicle solubilization. For vesicle solubilization kinetics, measurements were conducted by adding a high concentration (ca. 5 mM) of the compound of the invention above its CMC to 100 μM POPC LUVs in a 3 mm×3 mm quartz glass cuvette. Final concentrations were 10.06 mM for 5a, 7.16 mM for 5b, 5.69 mM for 5c, 8.62 mM for 8a and 5.46 mM for 8c. Measurements were started immediately after mixing the vesicle suspension to monitor changes in light scattering intensity, I_{scatter} at an angle of 90° and at 25° C.

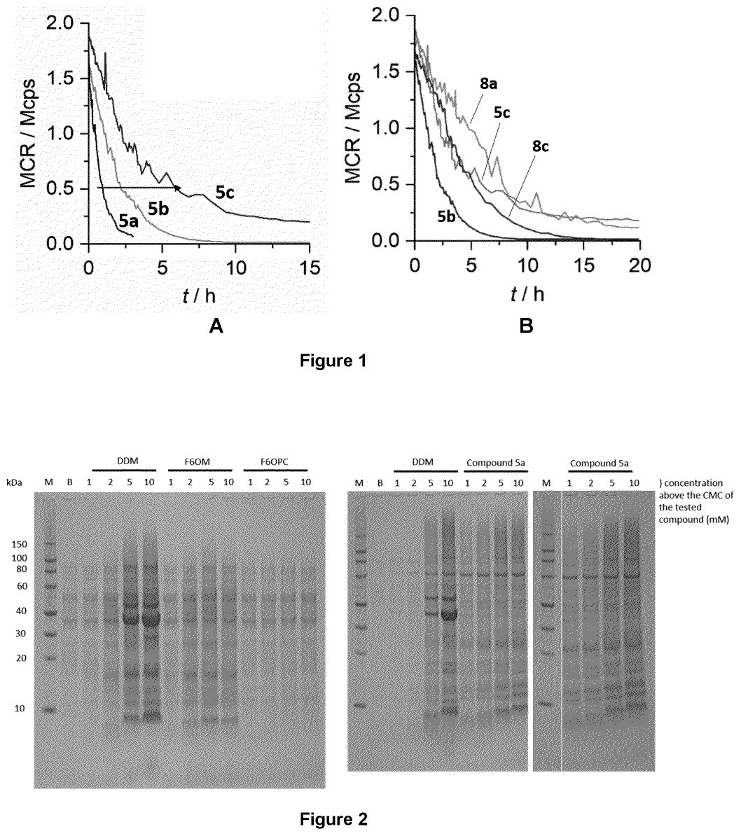

Results. The solubilization of preformed large unilamellar vesicles (LUVs) composed of the singly unsaturated phospholipid 1-palmitoyl-2-oleyl-sn-glycero-3-phosphocholine (POPC) was investigated. Measurements were conducted at 25° C. by adding a rather high concentration (CMC+ca. 5 mM) of the compounds of the invention to 100 μM POPC LUVs, which resulted in a steady decrease in the light scattering intensity over time. Solubilization was fast and complete (˜2 h) for compound 5a but took longer for compounds 5b and 5c (FIG. 1A). A trend is observed between the length of the hydrogenated chain and kinetics of solubilization, the longer the spacer the slower the solubilization. The potency of compounds 8a and 8c to solubilize LUV was also observed, however, a longer time was needed to achieve a complete solubilization (FIG. 1B).

### Example 3: Extraction of Membrane-Proteins from Native E. coli Membranes Using the Compounds of the Invention

Solubilization of Membrane-Proteins from native E. coli membranes. E. coli BL21(DE3) cells were transformed with an empty pET-24 vector and selected by kanamycin resistance. After incubation in 400 mL lysogeny broth overnight at 37° C. under constant agitation (150 rpm), cells were harvested by centrifugation and washed twice with saline (154 mM NaCl). Cell pellets were resuspended in ice-cold buffer (100 mM Na₂CO₃, pH 11.5) and subjected to ultrasonication in an S-250A sonifier (Branson Ultrasonics, Danbury, USA) twice for 10 min each. To remove cell debris, the lysate was centrifuged at 4° C. for 20 min at 7149 rpm (3000 g). The supernatant was ultracentrifuged at 4° C. for 1 h at 31 400 rpm (100,000 g) to separate membrane fragments from soluble and peripheral proteins. Membrane pellets were washed and suspended in working buffer, ultracentrifuged again at 4° C. for 1 h at 31 400 rpm (100,000 g) to remove any residual soluble or peripheral proteins. The resulting pellets were resuspended in buffer (50 mM Tris, 200 mM NaCl, pH 7.4) to a final concentration of 100 mg wet-weight pellet per 1 mL of buffer and mixed in a 1:1 volume ratio with stock solutions of the compounds of the invention in buffer. All samples were incubated for at least 16 h at 20° C. under constant, gentle agitation (500 rpm) and subsequently ultracentrifuged at 4° C. for 1 h at 51 000 rpm (100,000 g). The solubilized supernatant containing micelles was analyzed using SDS-PAGE.

Sodium dodecyl sulphate polyacrylamide gel electrophoresis (SDS-PAGE). The solubilization efficiency of compound 5a, F₆OM, F₆OPC and DDM on biological membranes was assessed by SDS-PAGE using a NuPAGE Bis-Tris system (Life Technologies, Carlsbad, USA) with a polyacrylamide gradient of 4-12%. 14-μL samples were mixed with 5 μL 4×SDS sample buffer (106 mM Tris HCl, 141 mM Tris base, 2% (w v) SDS, 10% (w v) glycerol, 0.51 mM EDTA, 0.22 mM SERVA Blue G250, and 0.175 mM Phenol Red, pH 8.5) and 1 μL 1 M dithiothreitol (DTT) and boiled at 95° C. for 10 min. 12 μL of each sample was loaded on a ready-to-use NuPAGE. As reference, a standard-weight marker (Roti-Mark 10-150, Carl Roth, Karlsruhe, Germany) was used, and the working buffer was used as negative control. Gel electrophoresis was performed for 45 min in MES buffer (50 mM MES, 50 mM Tris base, 0.1% (w v) SDS, 1 mM EDTA) at 200 V and 50 W. Subsequently, gels were fixed for 20 min (10% (w v) acetic acid, 40% (w v) ethanol), stained for 30 min (0.025% (w v) Coomassie brilliant blue G250, 10% (w v) acetic acid) and destained overnight in water. For quantification of solubilization efficiencies, gels were photographed with a C₄₀₀₀Z camera (Olympus, Tokyo, Japan), and protein bands were analyzed with ImageJ software.

This experiment was carried out at concentrations of 1, 2, 5, and 10 mM above the CMC of the tested compound (CMC+1 mM, CMC+2 mM, CMC+5 mM, CMC+10 mM).
CMC of DDM: 0.17 mM (VanAken, et al. (1986) Methods Enzymol. 125, 27-35).
CMC of F₆OM: 0.7 mM (Frotscher, E., et al. (2015) Angew. Chem. Int. Ed. 54, 5069-5073).
CMC of F₆OPC: 2.9 mM (Frotscher, E., et al. (2015) Angew. Chem. Int. Ed. 54, 5069-5073).

Results. The intensities (i.e., pixel counts) of SDS-PAGE band patterns (FIG. 2) were analyzed to provide the overall protein-extraction yields which were expressed relative to the buffer without any detergent. FIG. 2 indicates that compound 5a efficiently extracted membrane-proteins from E. coli membranes size range. Notably, at low concentrations (i.e., CMC+1-5 mM), compound 5a displayed better solubilization efficiencies than DDM, although DDM was highly efficient in extracting a single abundant protein of ˜35 kDa, namely, outer-membrane protein OmpA, at higher concentrations. By contrast F₆OM and F₆OPC showed very limited solubilization. This indicates that, at any concentration, the compound of the invention (compound 5a) allow to obtain a higher membrane-protein extraction yield than F₆OM and F₆OPC.

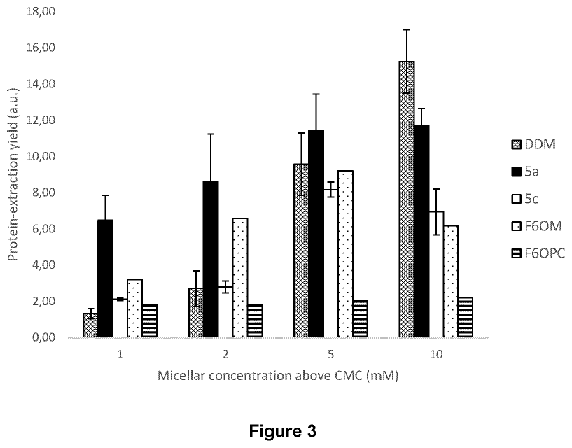

FIG. 3 also shows that the performances of compound 5c are similar to those of DDM at low concentrations (CMC+1-5 mM).

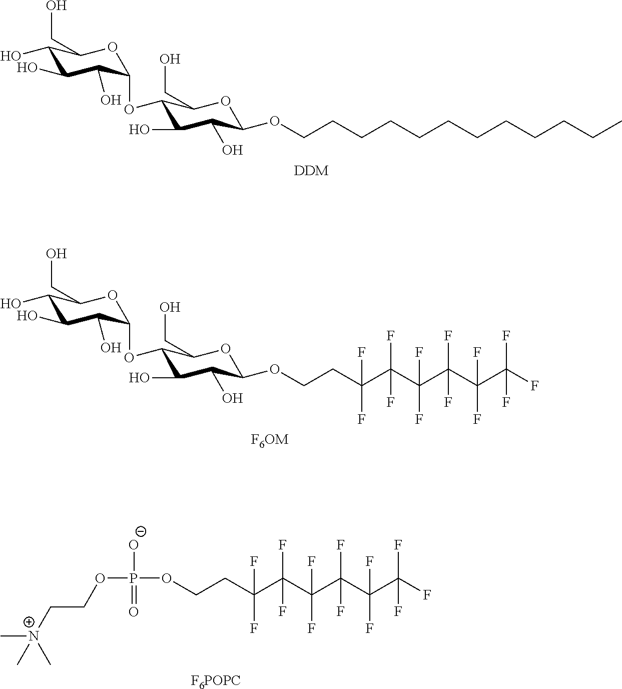

1-16. (canceled)

17. A compound represented by the following formula (I): 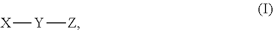
wherein:
X is a polar moiety selected from a maltoside, a lactobionamide, a glucoside, a sulfobetaine, an aminoxide, a sulfate, and a branched diglucoside moiety;
Y is a (C₁-C₁₂) aliphatic linker, optionally comprising one or more heteroatomic groups selected each independently from —O—, —NH—, —S—, —C(O)—, —NH—C(O)—, —C(O)—NH—, —C(O)—O—, —O—C(O)—, —NH—C(O)—O—, —O—C(O)—NH— and triazole; and
Z is selected from a perfluorinated (C₃-C₁₂)cycloalkyl and a branched perfluorinated (C₃-C₁₂)alkyl.

18. The compound according to claim 17, wherein X is a maltoside, a lactobionamide or a sulfobetaine moiety.

19. The compound according to claim 17, wherein:
X is a maltoside or lactobionamide moiety;
Y is a (C₁-C₁₂)aliphatic linker; and
Z is selected from a perfluorinated (C₃-C₁₂)cycloalkyl and a branched perfluorinated (C₃-C₁₂)alkyl.

20. The compound according to claim 17, wherein Y is a (C₃-C₈)alkylene linker.

21. The compound according to claim 17, wherein Y is selected from a propylene, a butylene and a pentylene.

22. The compound according to claim 17, wherein Z is a perfluorinated (C₃-C₁₂)cycloalkyl or a a perfluorinated (C₅-C₇)cycloalkyl.

23. The compound according to claim 17, wherein Z is a perfluorinated cyclohexyl.

24. The compound according to claim 17, wherein Z is a perfluorinated isoheptyl or a perfluorinated isopentyl.

25. The compound according to claim 17, wherein:
X is a maltoside, a lactobionamide or a sulfobetaine moiety;
Y is a (C₃-C₈)alkylene linker; and
Z is a perfluorinated cyclohexyl.

26. The compound according to claim 17, wherein said compound is of formula (II): 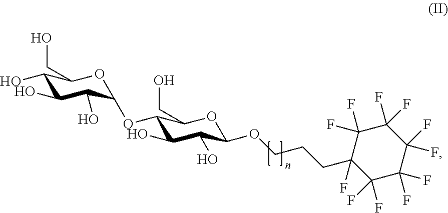
in which n is 1, 2 or 3.

27. The compound according to claim 17, wherein said compound is of formula (III): 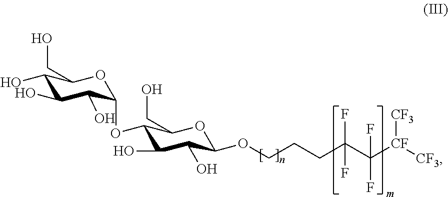
in which n is 1, 2 or 3, and m is 1 or 2.

28. The compound according to claim 17, wherein said compound is selected from the group consisting of:
3-(perfluorocyclohexyl)-propoxy-4-O—(α-D-glucopyranosyl)-β-D-glucopyranoside,
4-(perfluorocyclohexyl)-butoxy-4-O—(α-D-glucopyranosyl)-β-D-glucopyranoside,
5-(perfluorocyclohexyl)-pentoxy-4-O—(α-D-glucopyranosyl)-β-D-glucopyranoside,
3-(perfluoroisopentyl)-propoxy-4-O—(α-D-glucopyranosyl)-β-D-glucopyranoside,
3-(perfluoroisoheptyl)-propoxy-4-O—(α-D-glucopyranosyl)-β-D-glucopyranoside,
5-(perfluoroisopentyl)-pentoxy-4-O—(α-D-glucopyranosyl)-β-D-glucopyranoside,
3-(perfluorocyclohexyl)-N-propane Octahydroxy lactobionamide, and
3-(dimethyl(5-(perfluorocyclohexyl)pentyl)ammonio)propane-1-sulfonate.

29. A detergent composition comprising at least one compound as defined in claim 17.

30. An in-vitro method for extracting a membrane-protein from a biological sample comprising the following steps:
(a) contacting a compound as defined in claim 17 of a detergent composition comprising said compound with a biological sample comprising a membrane-protein; and
(b) recovering a membrane-protein.

31. The method according to claim 30, wherein the membrane-protein is Bacteriorhodopsin or FhuA.
